(19) United States
(12) Patent Application Publication
(10) Pub. No.: US 2025/0262276 A1
(43) Pub. Date: Aug. 21, 2025

(54) METHODS OF INHIBITING BOVINE MASTITIS DURING THE DRY PERIOD

(71) Applicant: ELANCO US INC., Greenfield, IN (US)

(72) Inventors: Joanna CAIN, Greenfield, IN (US); Mike STEELE, Greenfield, IN (US); Wendela WAPENAAR, Greenfield, IN (US)

(21) Appl. No.: 18/566,294

(22) PCT Filed: Jun. 2, 2022
(86) PCT No.: PCT/US2022/032002

§ 371 Date: Dec. 1, 2023

Related U.S. Application Data
(60) Provisional application No. 63/196,040, filed on Jun. 2, 2021.
(60) Provisional application No. 63/301,858, filed on Jan. 21, 2022.

(51) Int. Cl. A61K 38/19; A61P 15/14; A61P 31/04

(52) U.S. Cl. CPC A61K 38/193; A61P 15/14; A61P 31/04

(57) ABSTRACT

The invention provides methods of treating and inhibiting mastitis in dairy cows, in need thereof, wherein said method comprises: administering pegbovigrastim to the cow during the late lactation stage and about the dry-off day.

### CROSS-REFERENCE TO RELATED APPLICATIONS

This application is a national stage entry under 35 U.S.C § 371 (b) of PCT International Application No. PCT/US2022/032002, filed Jun. 2, 2022, which claims priority under 35 U.S.C. § 119 (e) to U.S. Provisional Application Ser. No. 63/196,040 filed on Jun. 2, 2021 and to U.S. Provisional Application Ser. No. 63/301,858 filed on Jan. 21, 2022, the entire disclosures of all of which are incorporated herein by reference.

### INCORPORATION BY REFERENCE OF MATERIAL SUBMITTED ELECTRONICALLY

Incorporated by reference in its entirety is a computer-readable Sequence Listing identified as follows: a 6 kilobyte.txt file named “78045_395184_ST25.txt”, created on Jul. 17, 2024.

### BACKGROUND OF THE INVENTION

Mastitis is an inflammation of the mammary gland (i.e. teats and udder), typically caused by infection. Mastitis causes large economic losses to the dairy industry. For instance, mastitis is estimated to cost the global dairy industry US$19.7 to US$32 billion annually (according to a 2016 study by the University of Glasgow). The U.S. alone estimates it loses US$2 billion to mastitis in dairy annually. Mastitis affects the profitability of a herd in a number of ways, both directly and indirectly, including: loss of milk production, higher culling rates of infected cows, decreased value of milk, discarded milk following antibiotic treatment and veterinary costs. Additionally, mastitis affects the performance, health, and welfare of the animal.

Although about 20 to 35% of clinical mastitis cases are of unknown etiology (Wellenberg et al., “Viral infections and bovine mastitis: a review.” Veterinary Microbiology. 2002; 88:27-45), it is widely accepted that bovine mastitis is mainly bacterial in origin. Mastitis can be classified as contagious or environmental (Blowey et al., “Mastitis control in dairy herds.” Farming Press (Ipswich) 1995. p. 29). Contagious mastitis is caused by organisms such as Staphylococcus aureus, Strep. dysgalactiae and Strep. agalactiae, which are all adapted to survive in the udder, causing subclinical infections.

The mammary gland of the dairy cow requires a nonlactating period prior to an impending parturition (i.e., calving) to optimize milk production in the subsequent lactation. This nonlactating period is called “the dry period”; and it includes the time between halting of milk removal (“dry-off”) and the subsequent calving. This period allows the regeneration of secretory tissue of the mammary gland. Although this period is critical for mammary gland remodeling, the cow is highly susceptible to new intramammary infections (IMI) during the early dry period (Dingwell et al., 2003). After drying-off, although milk is not being removed, the mammary gland temporarily continues to synthesize milk, which accumulates in the udder. The resulting increase in mammary pressure may cause leakage of milk via the teats, allowing microorganisms to gain entry into the mammary gland. In addition, at the beginning of involution, mammary gland secretions contain low concentrations of natural protective factors, such as immune cells, immunoglobulins, and lactoferrin, as well as high concentrations of fat, casein, lactose, and citrate, which can interfere with the defense capacity of the gland and provide an excellent medium for bacterial growth (Oliver and Sordillo, 1989; Collier et al., 2012). Once involution is completed, within 30 days after cessation of milking, the mammary gland becomes much more resistant to new IMI because of a low fluid volume in the udder and a medium unfavorable for bacterial growth (Burvenich et al., 2007). With increasing milk production, drying-off has become a challenging period for the dairy cow. Rajala-Schultz et al. (2005) established that the risk of IMI at calving increases by 77% for every 5 kg of milk produced above 12.5 kg when milking is stopped.

Because mastitis-causing microbes accumulate on teat ends shortly after dry-off, attempts to reduce these microbe populations have been made by conventional “dry cow therapy.” Such therapy includes teat dipping and the use of antibiotics during the early dry period. However, the results of such therapies have been mixed. For example, one investigation showed that dipping teats in a 5% tincture of iodine at dry-off, and again 24 hours later, significantly reduced new S. aureus infections but not those caused by Strep. uberis. And, no protection was found by dipping daily using a 1% iodine dip for 7 days after dry-off. Additionally, although infusion of the udder with antibiotics can help prevent infections that occur in the early dry period, there is a risk of the development of antibiotic resistant microorganisms. For example, present treatments are not effective against all species of bacteria, such as coliforms which develop resistant strains. Further, elimination of common udder pathogens, such as Staphylococcus species and Corynebacterium bovis via treatment, may render cows more susceptible to less common pathogens. Teat sealants have shown better efficacy but further studies are needed to investigate their effect on milk somatic cell counts in lactating dairy cows (Rabiee et al., “The effect of internal teat sealant products (Teatseal and Orbeseal) on intramammary infection, clinical mastitis, and somatic cell counts in lactating dairy cows: a meta-analysis”, J Dairy Sci. 2013; 96 (11): 6915-6931.)

Clearly, there remains a need for an effective method to decrease the incidence of infections in dairy cows during the dry period, especially treatments that are effective against a wide spectrum of microorganisms (including, e.g., coliforms, staphylococcal species and streptococcal species), and that does not have the environmental disadvantages of antibiotics.

### BRIEF DESCRIPTION OF THE DRAWINGS

FIG. 1: Study results show that Imrestor® significantly increased both WBC and neutrophil counts.

FIG. 1A: Study results show plasma DiHomo gamma linolenic acid by treatment and time.

FIG. 1B: Study results show plasma eicosapentaenoic acid by treatment and time.

FIG. 2: Study results show plasma TXB2 by treatment and time.

FIG. 3: Study results show serum reactive oxygen species.

FIG. 4: Study results show serum antioxidant potential.

FIG. 5: Study results show oxidative stress index.

FIG. 6: Study results show serum glucose by treatment and time.

FIG. 7: Study results show serum calcium by treatment and time.

FIG. 8: Study results show serum haptoglobin by treatment and time.

FIG. 9: Study results show total leukocyte count by treatment and time.

FIG. 10: Study results show neutrophil count by treatment and time.

FIG. 11: Study results show lymphocyte count by treatment and time.

FIG. 12: Study results show albumin concentrations in mammary gland secretions by treatment and time during involution.

FIG. 13: Study results show lactoferrin concentrations in mammary gland secretions by treatment and time during involution.

FIG. 14: Study results show alpha-lactalbumin concentrations in mammary gland secretions by treatment and time during involution.

FIG. 15: Study results show number of quarters with IMI by collapsed sampling period.

FIG. 16: Study results show effect of treatment on somatic cell count (log) across sampling period using-7 days before drying off as a covariate. (A=Control; B=Imrestor®).

FIG. 17: Study results show effect of treatment on daily milk production (kg) across sampling period using-7 days before drying off and parity as covariate. (A=Control; B=Imrestor®).

### SUMMARY OF THE INVENTION

In one aspect, the invention provides a method of treating mastitis in a cow, in need thereof, wherein said method comprises: administering pegbovigrastim to the cow during the late lactation stage. In one embodiment, pegbovigrastim is administered to the cow about 5, 6, 7, 8, 9, 10, 14, 20, 25 or 30 days before the dry-off day. In one embodiment, pegbovigrastim is administered 7 days before the dry-off day. In one embodiment, the method further comprises administering pegbovigrastim to the cow on the dry-off day. In one embodiment, pegbovigrastim is administered to the cow about 5, 6, 7, 8, 9, 10, 14, 20, 25 or 30 days after the dry-off day. In one embodiment, the mastitis is subclinical mastitis. In one embodiment, antibiotics are not administered. In one embodiment, the dose of pegbovigrastim is about 2-40 μg/kg. about 10-40 μg/kg, about 20-40 μg/kg. about 30-40 μg/kg, about 20-30 μg/kg. 2-10 μg/kg, or about 10-20 μg/kg, based on the weight of the cow. In one embodiment, the dose of pegbovigrastim is about 20-40 μg/kg, based on the weight of the cow.

In one aspect, the invention provides a method of inhibiting mastitis in a cow, in need thereof, wherein said method comprises: administering pegbovigrastim to the cow during the late lactation stage. In one embodiment, pegbovigrastim is administered to the cow about 5, 6, 7, 8, 9, 10, 14, 20, 25 or 30 days before the dry-off day. In one embodiment, pegbovigrastim is administered 7 days before the dry-off day. In one embodiment, the method further comprises administering pegbovigrastim to the cow on the dry-off day. In one embodiment, pegbovigrastim is administered to the cow about 5, 6, 7, 8, 9, 10, 14, 20, 25 or 30 days after the dry-off day. In one embodiment, the mastitis is subclinical mastitis. In one embodiment, antibiotics are not administered. In one embodiment, the dose of pegbovigrastim is about 2-40 μg/kg, about 10-40 μg/kg, about 20-40 μg/kg, about 30-40 μg/kg, about 20-30 μg/kg, 2-10 μg/kg, or about 10-20 μg/kg, based on the weight of the cow. In one embodiment, the dose of pegbovigrastim is about 20-40 μg/kg, based on the weight of the cow. In one embodiment, the method reduces the incidence of mastitis by an amount greater than about 10%, when compared to a cow that was not administered pegbovigrastim. In one embodiment, the method reduces the incidence of mastitis in an amount of from about 40% to about 100%, when compared to a cow that was not administered pegbovigrastim. In one embodiment, the method reduces the incidence of mastitis in an amount of about 50% when compared to a cow that was not administered pegbovigrastim. In one embodiment, subclinical mastitis is inhibited from developing into clinical mastitis.

In one aspect, the invention provides a method of increasing milk production in a dairy cow, wherein said method comprises: administering pegbovigrastim to the cow during the late lactation stage. In one embodiment, pegbovigrastim is administered to the cow about 5, 6, 7, 8, 9, 10, 14, 20, 25 or 30 days before the dry-off day. In one embodiment, pegbovigrastim is administered 7 days before the dry-off day. In one embodiment, the method further comprises administering pegbovigrastim to the cow on the dry-off day. In one embodiment, pegbovigrastim is administered to the cow about 5, 6, 7, 8, 9, 10, 14, 20, 25 or 30 days after the dry-off day.

In one aspect, the invention provides a method of treating mastitis in a cow, in need thereof, wherein said method comprises: administering pegbovigrastim to the cow in two doses: i) at about a week before the expected calving day, and ii) within about 24 hours after calving. In one embodiment, pegbovigrastim is administered to the cow about 5 to 10 days before the expected calving day. In one embodiment, pegbovigrastim is administered 7 days before the expected calving day. In one embodiment, pegbovigrastim is administered within about 20-30 hours after calving. In one embodiment, the mastitis is subclinical mastitis. In one embodiment, antibiotics are not administered. In one embodiment, the dose of pegbovigrastim is about 2-40 μg/kg, about 10-40 μg/kg, about 20-40 μg/kg, about 30-40 μg/kg, about 20-30 μg/kg, 2-10 μg/kg, or about 10-20 μg/kg, based on the weight of the cow. In one embodiment, the dose of pegbovigrastim is about 15 mg.

In one aspect, the invention provides a method of inhibiting mastitis in a cow, in need thereof, wherein said method comprises: administering pegbovigrastim to the cow in two doses: i) at about a week before the expected calving day, and ii) within about 24 hours after calving. In one embodiment, the method reduces the incidence of mastitis in an amount of from about 40% to about 100%, when compared to a cow that was not administered pegbovigrastim.

In one aspect, the invention provides a method of increasing milk production in a dairy cow, wherein said method comprises: administering pegbovigrastim to the cow in two doses: i) at about a week before the expected calving day, and ii) within about 24 hours after calving. In one embodiment, the method increases milk production by about 5%, about 10%, about 15%, or about 20%, when compared to a cow that was not administered pegbovigrastim.

### DETAILED DESCRIPTION OF THE INVENTION

The present invention includes methods of treating, and inhibiting, mammary gland inflammation typically caused by infections, particularly infections occurring at, or developing during, a critical stage of a mammal's lactation cycle, i.e., the dry period, in a mammal in need thereof. A mammal in need thereof is any mammal at risk of contracting mammary gland inflammation/infection, or has mammary gland inflammation/infection.

### Mastitis

Mastitis is inflammation of the mammary gland (e.g., intramammary gland inflammation). Mastitis can affect any mammal, for example cows, ewes, and goats. Mastitis is mainly caused by gram-positive and gram-negative bacterial infections, and especially affects cows in intensive milk producing units.

Some of the main pathogenic microorganisms causing bovine mastitis are Staphylococcus aureus, Streptococcus agalactiae, Streptococcus uberis, Streptococcus dysgalactiae, Escherichia coli, Aerobacter aerogenes, Klebsiella pneumoniae, and Pseudomonas aeruginosa. These microorganisms invade the udder through the teat canal and produce inflammation of the milk-producing tissue potentially causing the formation of scar tissue which, once formed, may cause a permanent reduction in the cow's milk production. An infection can also alter the composition, quantity, appearance and quality of the milk. Mastitis-causing pathogens fall into two categories, namely, contagious and environmental. Contagious bacteria, such as Streptococcus agalactiae and Staphylococcus aureus, primarily colonize host tissue sites such as mammary glands, teat canals, and teat skin lesions; and are spread from one infected cow to another during the milking process. Environmental bacteria, often Streptococci, Enterococci, and Coliform organisms, are commonly present within the cow's surroundings from sources such as cow feces, soil, plant material, bedding, or water; and infect by casual opportunistic contact with an animal. In all cow mastitis cases, whatever the causal microorganism, the route of transmission of the invading pathogen into the inner gland of the udder is through the teat orifice and teat canal.

Mastitis may exist in a subclinical form in which there is no swelling of the gland or any observable abnormality of the milk, although there are changes in the milk that can be detected by specific tests. Subclinical mastitis can develop into clinical mastitis in which the abnormal conditions of the udder and secretion are observable. In this specification, reference to “mastitis” may include all forms of mastitis.

The presence of subclinical mastitis can be determined by measuring Somatic Cell Count (SCC) in a cow's milk. In particular, SCC is an accepted standard to assess inflammation in lactating mammary glands. Primarily, SCC is composed of leukocytes, or white blood cells, that are produced by the cow's immune system to fight an inflammation in the mammary gland (i.e., mastitis). Somatic cell count (SCC) is the total number of cells per milliliter in milk. The concentration of these cells in milk from uninfected/uninflamed mammary quarters (i.e., “normal milk”) is less than about 100,000 cells/ml, based on twice daily milking at regular intervals. A quarter is designated as having subclinical mastitis if the milk SCC is equal to or exceeds 200,000 cells/ml, in the absence of clinical changes (i.e., the quarter is likely to be infected, and the milk has reduced manufacturing properties such as reduced shelf life of fluid milk, and reduced yield and quality of cheese). A typical method for counting milk SCC is the fluoro-opto-electronic method (Bulletin IDF No. 321, pp. 39, 1996) using either a Fossomatic or Bentley machine.

Somatic Cell Counts of 100,000 to 199,999 cells/ml represent a range that is difficult to attribute to inflammation and/or intramammary infection. However, milk produced by cow with observable inflammation on a cow's quarter (i.e., clinical mastitis) is, by definition, “abnormal milk” and no reference to SCC is required.

In addition to SCC, whether a cow has subclinical mastitis can be assessed by the visual appearance of the produced milk. Milk from infected mammary glands contain flakes, clots, or other gross alterations in appearance. Such abnormalities are indicators of milk that is unsuitable for human consumption. In general, the more severe the infection, the greater the abnormal appearance of the secretion from the infected quarter.

### The Lactation Cycle

Cows must calve to produce milk and the lactation cycle is the period between one calving and the next. The cycle is split into four phases, the early, mid, and late lactation (each of about 120 days), and the dry period (typically about 45-60 days; however, the dry period can be up to 120 days long). During the dry period, the cow is not lactating. Having a dry period optimizes milk production in the subsequent lactation. Milk production would be 25-30% less in the subsequent lactation if a dry period were not allowed. The dry period ends at parturition.

The normal procedure to commence the dry period is to “dry off” a cow. The “dry off” procedure involves transitioning a cow to a low energy ration (e.g., shifting to a lower quality feed and/or providing fewer calories). Such transition reduces the milk production several days before the start of the dry period. The dry period begins on the “dry-off day” which is the day when milking is halted. That is, “dry-off day” is the first day of the dry period. Once milking is halted, increases in the intramammary pressure and accumulation of milk products in the gland inhibit further milk secretion.

The primary function of the mammary gland during lactation is one of continuous synthesis and secretion of large quantities of milk. The physiology of the udder during the dry period differs markedly from that during lactation. During the dry period, changes occur in the mammary gland which influence mammary cell proliferation and mammary function in the subsequent lactation. In particular, active milk-producing cells regress to a nonsecretory, resting state to prepare for the next lactation. During the dry period, the mammary gland progresses through three distinct stages: (1) “active involution”; (2) “steady state involution”; and (3) “colostrum formation”. During “active involution,” the mammary gland is highly susceptible to new intramammary infections since it is undergoing physiological changes and is more exposed to bacteria from the environment because the keratin plug is not fully developed, and bacteria do not get flushed out of the streak canal as during a milking process. (In contrast, during steady state involution, the gland is very resistant to infection due to an increase in activity of antibacterial factors in lacteal secretions. During colostrogenesis, as the mammary gland tissues transition to those synthesizing and secreting copious quantities of lacteal fluids, susceptibility to infection again increases.)

The “active involution” stage begins on the dry-off day and is completed by approximately three to four weeks into the dry period. (The period of steady state involution does not have a distinct beginning or end; the length is proportional to the length of the dry period. Colostrum formation begins one to three weeks prepartum and is characterized by the development of milk-producing cells and onset of copious milk secretion.)

Bovine Granulocyte-Colony Stimulating Factor (bG-CSF) Polypeptide

The methods of the present invention comprise administering an effective amount of a bovine granulocyte-colony stimulating factor (bG-CSF) polypeptide to a dairy cow at specific times during the cow's lactation cycle to treat, and/or inhibit, mammary gland infections (e.g., mastitis).

Bovine granulocyte colony stimulating factor is an endogenous protein that enhances neutrophil bactericidal functions and increases the production of neutrophils from bone marrow precursors. An “effective amount” is an amount which will relieve to some extent at least one of the symptoms of a mammary gland infection/inflammation, or inhibit a mammary gland infection/inflammation. Compositions containing a bG-CSF polypeptide can be administered for prophylactic, enhancing, and/or therapeutic treatments.

Preferably, the bG-CSF polypeptides of the present invention comprise at least one non-naturally-encoded amino acid. Examples of such bG-CSF polypeptides are disclosed in U.S. Pat. No. 10,138,283; the subject matter of such patent is incorporated herein by reference in its entirety. A preferred example is pegbovigrastim, which is a recombinant bG-CSF covalently bound to polyethylene glycol. Pegbovigrastim has the trade name Imrestor®, marketed by Elanco Animal Health.

The sequence of pegbovigrastim is as follows:

(SEQ ID NO: 1)
TPLGPARSLP QSFLLKCLEQ VRKIQADGAE LQERLCAAHK
LCHPEELMLL RHSLGIPQAP LSSCSSQSLQ LTSCLNQLHG
GLFLYQGLLQ ALAGISPELA PTLDTLQLDV TDFATNIWLQ
MEDLGAAPAV QPFQGAMPTF TSAFQRRAGG VLVASQLHRF
LELAYRGLRY LAEP

(Disulfide bridge: 36-42, 64-74; Modified residue: 133 F=4-(methoxyPEGcarbonylamino-ethoxyiminoethyl).)

### CAS: 1363409-60-2; PubChem: 172232540

The early dry period (i.e., the “active involution” stage) is a critical juncture in determining mammary gland health and milk production for the subsequent lactation. This stage of the dry period is characterized by dramatic alterations in metabolism, highly orchestrated immune responses, and changes to oxidant status. One of the initial immune responses to involution include recruitment of neutrophils to the mammary gland. Without wanting to be held to a mechanism of action, it is believed that pegbovigrastim optimizes mammary involution due to its capacity to increase circulating neutrophils.

### Inhibition of Mastitis

The acquisition of mammary infections during the dry period has a dramatic impact on the incidence of clinical mastitis in the subsequent lactation. For example, dairy cows can acquire a sub-clinical form of mastitis during the dry period which develops into clinical mastitis during the subsequent lactation.

In one embodiment, an effective amount of a bG-CSF polypeptide (e.g., pegbovigrastim) is administered to a dairy cow at specific times of the lactation cycle to inhibit mastitis, e.g., prevent the development of mastitis, especially inhibit mastitis in the subsequent lactation.

In the present specification, the term “inhibit” includes “reduce the likelihood of contracting” and/or “prevent.” That is, the methods of the present invention are considered to be effective if they reduce the likelihood of, or prevent, any symptom associated with mastitis.

Inhibition of symptoms can be assessed by comparing the incidence of mastitis of different subjects exposed to the same environment (e.g., the same intense milking farm), wherein some subjects are administered a bG-CSF polypeptide and some subjects are not administered a bG-CSF polypeptide.

In one embodiment, the incidence of mastitis in cows administered pegbovigrastim is reduced by an amount greater than about 10%, when compared to cows that were not administered pegbovigrastim. In another embodiment, the incidence of mastitis is reduced in an amount of from about 40% to about 100%, when compared to cows that were not administered pegbovigrastim. In a further embodiment, the incidence of mastitis is reduced in an amount of about 50% when compared to cows that were not administered pegbovigrastim.

### Treatment of Mastitis

In another embodiment, an effective amount of a bG-CSF polypeptide (e.g., pegbovigrastim) is administered to a dairy cow to treat mastitis. In the present specification, the term “treat” includes “reduce the severity of a symptom” and/or “shorten duration” of mastitis. That is, the methods of the present invention are considered to be effective if they reduce any symptom associated with mastitis and/or shorten the duration of an episode of any such symptom. In one embodiment, subclinical mastitis is treated during the dry period.

In one embodiment, the symptoms/duration of mastitis in cows administered pegbovigrastim is reduced by an amount greater than about 10%, when compared to cows that were not administered pegbovigrastim. In one embodiment, the symptoms/duration of mastitis in cows administered pegbovigrastim is reduced by an amount greater than about 25%, when compared to cows that were not administered pegbovigrastim. In another embodiment, the symptoms/duration of mastitis is reduced in an amount of from about 40% to about 100%, when compared to cows that were not administered pegbovigrastim. In a further embodiment, the symptoms/duration of mastitis is reduced in an amount of about 50% when compared to cows that were not administered pegbovigrastim.

### Increasing Milk Production

In a further embodiment, an effective amount of a bG-CSF polypeptide (e.g., pegbovigrastim) is administered to a dairy cow to increase milk production (i.e., milk yield) in the subsequent lactation. Milk yield may be assessed by daily milk weight values. In one embodiment, the milk production in cows is increased by an amount greater than about 5%, when compared to cows that were not administered pegbovigrastim. In one embodiment, the milk production in cows is increased by an amount greater than about 10%, when compared to cows that were not administered pegbovigrastim. In one embodiment, the milk production in cows is increased by an amount greater than about 15%, when compared to cows that were not administered pegbovigrastim. In another embodiment, the milk production in cows is increased in an amount of from about 10% to about 50%, when compared to cows that were not administered pegbovigrastim. In another embodiment, the milk production in cows is increased in an amount of from about 10% to about 25%, when compared to cows that were not administered pegbovigrastim. In another embodiment, the milk production in cows is increased in an amount of from about 5% to about 15%, when compared to cows that were not administered pegbovigrastim. In a further embodiment, the milk production in cows is increased in an amount of from about 25% when compared to cows that were not administered pegbovigrastim.

Administration of a bG-CSF Polypeptide

In one embodiment, a bG-CSF polypeptide is administered to a dairy cow in a single dose around dry-off day. For example, a single dose of pegbovigrastim is administered to a dairy cow in the time period ranging from about 2 days before dry-off day to about 2 days after dry-off day. For example, pegbovigrastim is administered on dry-off day.

In another embodiment, a bG-CSF polypeptide is administered to a dairy cow in a single dose during the late lactation phase of the lactation cycle. For example, pegbovigrastim is administered from about 1-3 weeks before dry-off day, typically about 5-10 days before dry-off day. For example, pegbovigrastim is administered about 7 days before dry-off day.

In another embodiment, a bG-CSF polypeptide is administered to a dairy cow in a single dose during the active involution stage of the dry period. For example, pegbovigrastim is administered from about 1-3 weeks after dry-off day, typically about 5-10 days after dry-off day. For example, pegbovigrastim is administered about 7 days after dry-off day.

In some embodiments, a bG-CSF polypeptide is administered to a dairy cow in two doses. In one embodiment, pegbovigrastim is administered: i) in the late lactation stage, and ii) around dry-off day. For example, pegbovigrastim is administered: i) about 1-3 weeks before dry-off day, typically about 5, 6, 7, 8, 9, 10, 11, 12, 13, or 14 days before dry-off day, and ii) in the period ranging from about 2 days before dry-off day to about 2 days after dry-off day (e.g., on dry-off day). For example, pegbovigrastim is administered: i) about 7 days before dry-off day, and ii) on dry-off day.

In one embodiment, a bG-CSF polypeptide is administered to a dairy cow in two doses: i) at around dry-off day, and ii) during the active involution stage of the dry period. For example, pegbovigrastim is administered: i) in a period ranging from about 2 days before dry-off day to about 2 days after dry-off day (e.g., on dry-off day), and ii) about 6, 7, 8, 9, 10, 11, 12, 13, or 14 days after dry-off day, typically about 5-8 days after dry-off day. For example, pegbovigrastim is administered: i) on dry-off day, and ii) about 7 days after dry-off day.

In one embodiment, a bG-CSF polypeptide is administered to a dairy cow in two doses: i) at about a week before the expected calving day, and ii) within about 24 hours after calving (i.e., parturition). For example, pegbovigrastim is administered: i) in a period ranging from about 5 to 10 days before the expected calving day, or 7 to 10 days before the expected calving day, and ii) within about 20-30 hours after calving.

In some embodiments, a bG-CSF polypeptide is administered to a dairy cow in multiple doses: i) at around dry-off day, and ii) during the late lactation phase; or i) at around dry-off day, and ii) during the active involution stage of the dry period. For example, three or four doses can be administered during these times.

In some embodiments, antibiotics are excluded when a bG-CSF polypeptide is administered in the methods of the present invention. Examples of typical antibiotics include beta-lactam drugs (including penicillin, ampicillin, amoxicillin, cloxacillin, cephapirin, and ceftiofur).

A bG-CSF polypeptide is administered to the bovine animal in any manner as would be known to a skilled artisan. In some embodiments, the compositions are administered enterally or parenterally (e.g., subcutaneously, intramuscularly, intravenously, by intra-dermal injection, as injectable solutions or suspensions, intraperitoneally, sublingually, and rectally (e.g., by suppositories)). Typically, pharmaceutical compositions comprising a bG-CSF polypeptide can further comprise a suitable carrier. Formulations suitable for parenteral administration include aqueous and non-aqueous sterile injection solutions that can contain anti-oxidants, buffers, bacteriostats and solutes that render the formulation isotonic with the blood of the recipient, and aqueous and non-aqueous sterile suspensions that can include suspending agents or thickening agents. In a preferred embodiment, the bG-CSF polypeptide is administered to the bovine animal by subcutaneous injection.

The actual preferred amounts of the polypeptide composition in a specified case will vary according to the particular compositions formulated, the mode of application, the particular sites of application, and the weight of the subject being treated.

Quantities herein are defined by ranges, and by lower and upper boundaries of ranges. Each lower boundary can be combined with each upper boundary to define a range. The lower and upper boundaries should each be taken as a separate element. Examples of typical dose amounts of pegbovigrastim to be administered by the methods of the present invention are from about 2 μg/kg to about 40 μg/kg, based on the weight of a dairy cow. Examples of other lower boundaries of this range include about 5 μg/kg, about 10 μg/kg, about 15 μg/kg and about 20 μg/kg. Examples of other upper boundaries of this range include about 25 μg/kg, about 30 μg/kg, about 35 μg/kg and about 38 μg/kg. In one embodiment, the dose is about 30 μg/kg for an average animal weight of 450-700 kg. Typically, such doses are used for dairy cows administered two doses, e.g., one during the late lactation phase and one around the dry-off day.

In some embodiments, a bG-CSF polypeptide is administered to a dairy cow at a dose of 2.7 mL/15 mg, or 1.35 mL/7.5 mg, or 0.68 mL/3.75 mg, administered in two doses, 7 days apart, i.e., 7 days before dry off and the day of dry off.

In one embodiment, pegbovigrastim is prepared in a concentration of about 4-7 μg/ml concentration, for example, about a 5.6 μg/ml concentration (e.g., prefilled syringe 15 mg in 2.7 ml). In one embodiment, presentations are in about 5, 10, 50, 100, and 200 ml multi-use vials.

### EXAMPLES

### Example 1

The objective of this study was to evaluate physiological changes in healthy early dry off cows, including how pegbovigrastim affects metabolic, immunologic, and redox changes that occur during the early dry period.

Late lactation cows (n=20) were matched by parity, milk production, BLV status, and SCC and randomly assigned to receive either 15 mg pegbovigrastim or saline 1 week prior to and on the day of dry-off. Blood samples were taken −7, −2, −1, 0, +1, +2, +4, +7, and +14 days relative to dry off as well as +5, +10, and +14 days post-parturition. Samples were analyzed for number of neutrophils, mononucleocytes, eosinophils, total calcium, BHB, NEFA, albumin, glucose, haptoglobin, reactive oxygen species (ROS), and antioxidant potential. Repeated measures models using PROC MIXED were used to assess the effects of treatment and means were separated using Bonferroni correction (SAS ver. 9.4).

Pegbovigrastim increased serum concentrations of neutrophils and mononucleocytes compared to control cows (P<0.001). There was a significant treatment and time by treatment effect of pegbovigrastim depressing serum glucose concentrations the 4 days post dry off (P<0.001). Pegbovigrastim tended to increase serum ROS concentrations while reducing serum calcium and haptoglobin concentrations (P<0.10) during the early dry period. Control cows had elevated BHB 14 days post-parturition (P<0.01). This study demonstrated pegbovigrastim injection at dry off had broad ranging effects on early dry cows which could influence health and production in the subsequent lactation.

### Example 2

### Randomized Clinical Trial Evaluating Effects of an Alternative Dosing Schedule for Pegbovigrastim on Mammary Gland Health and Milk Production

The objective of this randomized clinical trial was to evaluate effects of an alternative dosing schedule for pegbovigrastim (PEG; Imrestor®, Elanco Animal Health) on mammary gland health and milk production. Pregnant late lactation cows were randomly assigned to receive treatment with 15 mg of PEG (n=10 cows) or a sham injection with saline (n=10) administered 7 d before dry-off and on the day of dry off (DRY). No antimicrobial therapy was administered at DRY. Quarter (QTR) milk samples were collected for bacteriological culture and somatic cell count (SCC) at 8 periods (7 and 2 d before DRY, DRY, 7 and 14 d after DRY, and 5, 10, and 14 d after calving). Daily milk yield in the subsequent lactation were evaluated on 10, 14, 30, 60, and 120 DIM. Chi-square analysis was used to assess the effect of treatment on incidence of intramammary infection (IMI) and multivariate modeling was used to determine effects of treatment on SCC and milk yield.

The incidence of IMI was greater for QTR of cows in the control group as compared to QTR of cows that received treatment (X2=6.3; P=0.006). Compared to cows receiving treatment, the odds of IMI were 7.5 times greater (95% CI: 1.5, 36.7) for QTR in the control group.

While the overall effect of treatment on SCC was not significant (P=0.23), significant effects were found for period and the interaction of treatment by period (P<0.01).

Greater Log 10 SCC were observed for treated cows 2 d before DRY (4.09 control; 4.68 PEG) and at DRY (4.12 control; 4.62 PEG). Significant effects of sampling period (P<0.001) and an interaction of treatment by sampling period (P=0.001) were observed for milk yield, and there was an overall tendency for treated cows to have greater milk yield in the subsequent lactation (P=0.09). Cows in the control group produced 45, 48, 52, 52, and 42 kg/cow/d at each sampling period. In contrast, cows in the treatment group produced 48, 51, 62, 58, and 50 kg/cow/d. Cows treated with PEG using an alternative dosing schedule had reduced incidence of IMI during the dry period and increased milk yield in the subsequent lactation.

### Example 3

### Imrestor® Pilot Neutrophil Study—WUR (NL)

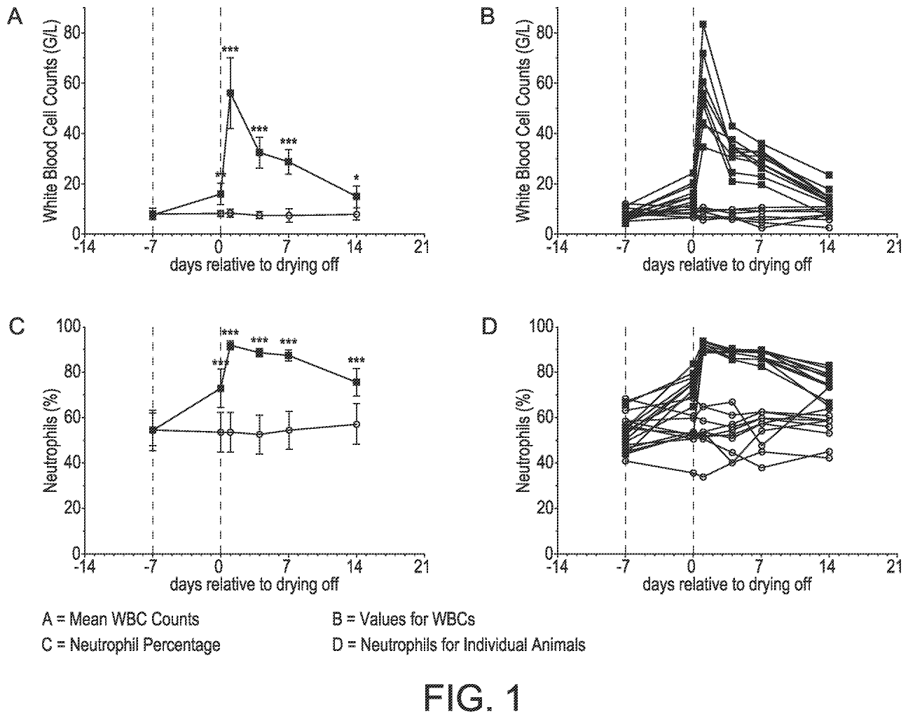

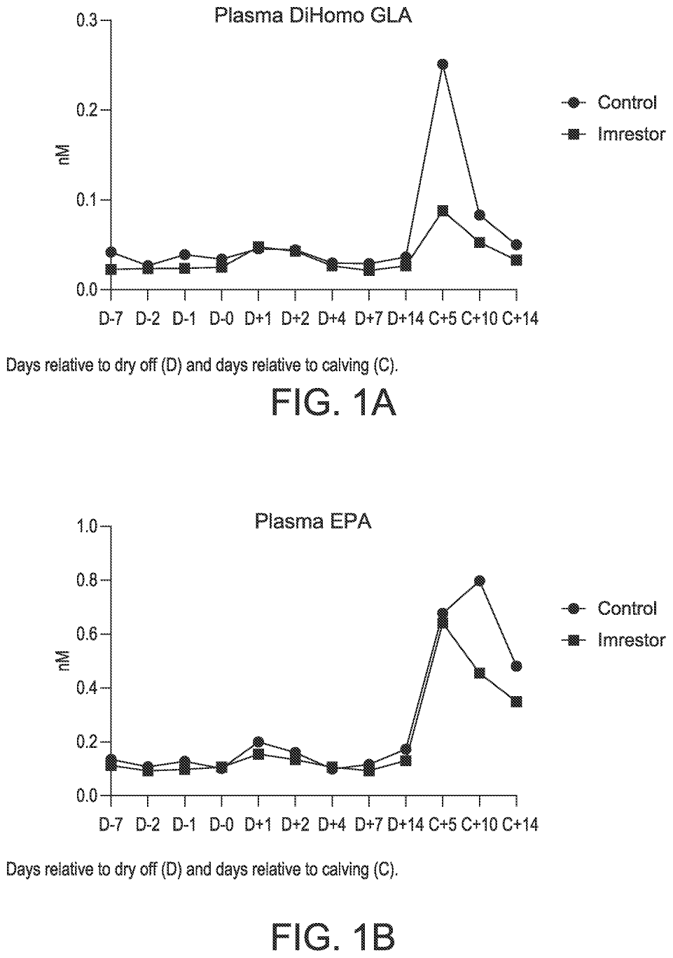

Objective: Investigation of the impact of Imrestor® on the incidence of mastitis (including assessment of neutrophil counts) during the dry period in dairy cows.
20 cows were in the study
two Groups: Control (saline) Group and Imrestor® Group
Enrolled animals were healthy, lactating, ready to be dried off approximately 6-9 weeks before expected calving date
Somatic cell count of most recent milk production recording before planned dry-off <150,000 cells/ml
2 Imrestor® injections were administered (day-7 and day 0 relative to dry-off day).
Mastitis and SCC (pre/post dry off) observed.
Functional assays of neutrophils performed: MPO production; Phagocytosis; Complete WBC. Results in FIG. 1 show WBC and Neutrophil counts.

The results show that administration of Imrestor® significantly increased both WBC counts and neutrophil counts. The increase was significant until the last time point, 14 d post drying-off.

### Example 4

### Imrestor® Pilot Field Study in Hungary

Objective: Test Effectiveness of Imrestor® in Reducing the Incidence of Naturally-occurring Clinical Mastitis in Dairy Cows during the Time of Dry-off until Return to Regular Milking days 4/5/6 postpartum

### Study Parameters:

154 cows/2 sites with high historical incidence of mastitis (20%)
Control (saline) and Imrestor® groups
2 injections on d-7 (i.e., 7 days before Dry-off day) and do (i.e., on Dry-off day) plus normal on farm Dry-off protocol (i.e., antibiotics and teat sealant)
Monitor cows for mastitis from d-7 until d3-5 post calving (i.e., 3 to 5 days after calving) for clinical mastitis
Perform CMTs (7 d prior to Dry-off and 4 d post calving)

Number of Animals (%)

| | | Removal due | | Removal for | |
|---|---|---|---|---|---|
| Treatment | Completed | to Mastitis | | Other Reasons | |
| Saline | 55 (71%) | 12 | (16%) | 10 | (13%) |
| Imrestor ® | 65 (84%) | 7 | (9%) | 5 | (6%) |

Table illustrates fate of the study animals. No mastitis cases at the time of dry-off until return to regular milking were observed. Mastitis after lactation onset: There was no statistical difference in the overall incidence of mastitis between the two treatment groups. However, overall incidence of mastitis was 9% for the Imrestor® group and 16% for the control group.

### Example 5

### MSU Product Testing of Imrestor® Administered Prior to Dry Off

### Results of Physiological Data

Brief Background: Objective was to determine how Imrestor® impacts involution when administered to dairy cattle around the time of drying off. Twenty cows at the MSU Dairy Teaching & Research Center were randomly assigned to receive Imrestor at −7 and day of dry off (Treatment—GROUP B) or received sham injections of saline (Control—GROUP A). Immunological and clinical outcomes were recorded throughout the dry period and post-calving. Cows did not receive any antibiotics at dry off and received an external sealant 14 days after—dry off (after dry period secretion sampling had been completed).

Outcome Variables Included in the study: Selected clinical and physiological outcomes (Table 1) during the predry, dry period and post-calving period were assessed. This report has results of physiological outcomes that measured involution and general health. Targeted polyunsaturated fatty acids (Table 2), were analyzed with LC-MS and 48 oxylipids (Table 3) were quantified using LC-MS/MS.

TABLE 1
Description of outcome variables and sampling periods.

| Sample | Outcome | Sampling Period | Status |
|---|---|---|---|
| Milk & | Isoprostanes; | Pre −7 d; Pre −2 d; Dry 0 d; Dry | Completed |
| Mammary | reactive oxygen | 7 d; Dry 14 d; Calv +5 d; | |
| secretions | species (ROS); | Calv +10 d; Calv +14 d | |
| | antioxidant | | |
| | potential (AOP) | | |
| | Albumin; lactoferrin; | Pre −7 d; Pre −2 d; Dry 0 d; Dry | |
| | citrate; α-lactalbumin | 7 d; Dry 14 d | |
| | Lactose | Pre −7 d; Pre −2 d; Dry 0 d; Dry | Assay unavailable |
| | | 7 d; Dry 14 d | due to COVID related |
| | | | supply chain issues |
| Blood samples | NEFA; BHB; glucose; | Pre −7 d; Pre −2 d; Dry −1 d; Dry0; | Completed |
| | calcium; albumin; | Dry +1; Dry +2; Dry +4; Dry +7; | |
| | WBC; haptoglobin; | Dry +14; Calv +5; Calv +10 d; | |
| | Isoprostanes; ROS, | Calv +14 d | |
| | AOP | | |

Analysis: All outcomes were evaluated in separate repeated measures models using Proc Mixed (SAS Vers 9.3). Models included fixed effects of treatment, time and the interaction of treatment x time. Cows was included in all models as a random term and as the subject for repeated measures.

Results: Polyunsaturated fatty acids in plasma and milk. A significant effect of time was found in all models (P<0.001) but none of the models included a significant treatment by time interaction (P>0.13). Only 2 models included a significant effect of treatment (none had a treatment by time interaction) but neither of those effects appeared meaningful (FIGS. 1a and b and Table 2).

DiHomo gamma linolenic acid (GLA) is a 20 carbon omega 6 fatty acid that is a desaturated and elongated product of linoleic acid and GLA, respectively. DiHomo GLA is a relatively uncommon fatty acid but does have biological significance with respect to its eicosanoid metabolites. Metabolism of DiHomo GLA via the cyclooxygenase (COX) 1 and COX2 pathways results in the series 1 thromboxanes and prostanoids that have known anti-inflammatory activities. In contrast, eicosanoids derived from the metabolism of arachidonic acid (AA) from the COX pathways results in the series 2 thromboxanes and prostanoids with potent pro-inflammatory effects. Interestingly, DiHomo GLA competes with AA for COX resulting in the inhibition of these pro-inflammatory series 2 thromboxanes and prostanoids. Eicosapentaenoic acid (EPA) is an omega-3 fatty acid derived from dietary sources or from the conversion of alpha-linolenic acid (ALA) through the action of desaturases and elongases. Metabolism of EPA results in the production of anti-inflammatory oxylipids. The reduction in both DiHomo GLA and EPA in the Imrestor group during the first weeks of lactation compared to the control group cannot be explained in terms of differences in oxylipid biosynthesis. Oxylipids derived from DiHomo GLA were not detected in any of the samples. The EPA-derived oxylipids (DiHETEs) were expressed in low amounts and were not different between treatment groups.

TABLE 2
Abbreviations and P-values for treatment for PFA found in
milk and sera (from a model including treatment, time and
treatment by time). Effect of Time P < 0.001 in all
models. Overall, no significant effects were found here.

| | | P-values for Treatment | |
|---|---|---|---|
| | | & Rx*Time | |
| | | Plasma | Milk |
| | | Rx effect; | Rx effect; |
| Polyunsaturated Fatty Acid | Abbreviation | Rx*Time | Rx*Time |
| Arachidonic acid | AA | 0.20; 0.82 | 0.58; 0.23 |
| α-Linolenic acid | ALA | 0.34; 0.70 | 0.21; 0.43 |
| Docosahexaenoic acid | DHA | 0.26; 0.96 | 0.21; 0.78 |
| Docosapentaenoic acid | DPA | 0.17; 0.38 | 0.25; 0.13 |
| Eicosapentaenoic acid | EPA | 0.04; 0.48 | 0.43; 0.33 |
| Dihomo-γ-linolenic acid | Dihomo-GLA | 0.02; 0.87 | 0.56; 0.21 |
| Linoleic acid | LA | 0.94; 0.85 | 0.59; 0.35 |

Results: Oxylipids and Isoprostanes: Assays were performed for a total of 48 lipid metabolites. Only 21 oxylipids and 2 isoprostanes were detected in plasma whereas 14 oxylipids and 2 Isoprostanes were found in milk (Table 3).

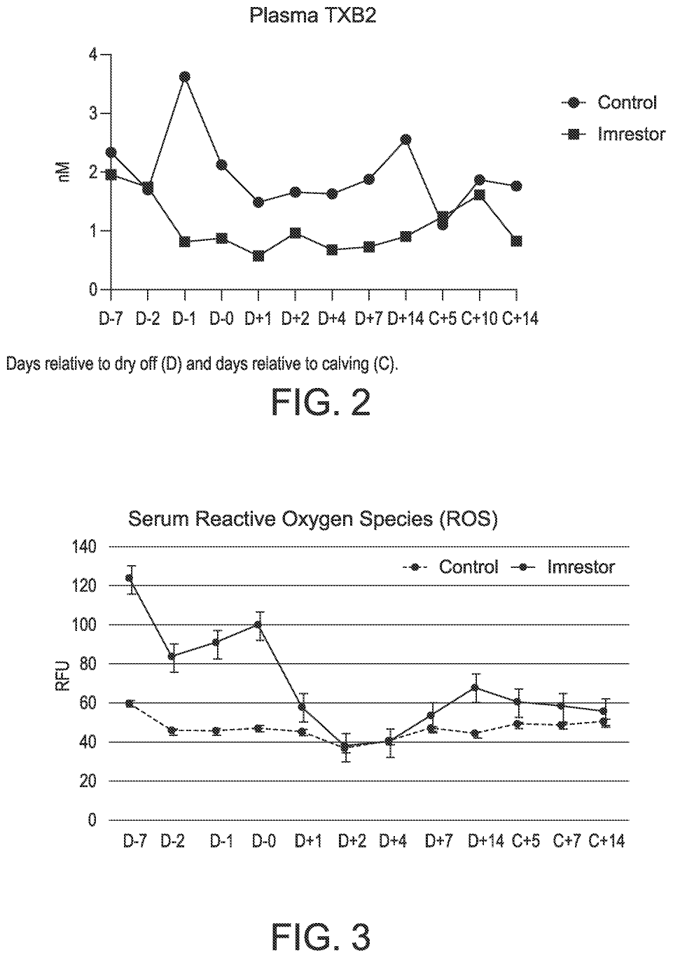

The only significant treatment effect among oxylipids and isoprostanes was with thromboxane B2 (TXB2) which is an inactive but stable metabolite/product of thromboxane A2 (TXA2) (FIG. 2, Table 4). Since TXA2 is very unstable in aqueous solution, it is hydrated immediately into the biologically inactive TXB2 that can be readily measured in biological solutions as a reflection of TXA2 production. As such, TXB2 is used routinely to assess TXA2 production. TXA2 is produced by activated platelets, endothelial cells, and macrophages. This thromboxane is generated from the metabolism of arachidonic acid through the cyclooxygenase pathway and through the enzymatic activity of thromboxane A synthase. TXA2 is known for its prothrombotic activity by activation of new platelets and increasing platelet aggregation. TXA2 is also a potent vasoconstrictor and is especially important during tissue injury and inflammation. Indeed, elevated TXA2 in humans has been associated with a number of diseases including cardiovascular diseases and is thought to negatively regulate immune responses. Finally, there is some evidence in human medicine that elevated blood concentrations of TXB2 is significantly related to oxidative stress (lipid peroxidation products) in type 1 diabetic patients. TXB2 was shown to be elevated in milk and sera of cows with mastitis and during times of oxidative stress (early dry off and around calving). Aspirin and other nonsteroidal anti-inflammatory drugs can be used to inhibit TXA2 activity.

Imrestor® clearly reduced TXB2 throughout most of the non-lactating period and at 14 days of lactation (FIG. 2). The inability to detect other prostanoids or to see any differences in isoprostane concentrations limit ability to speculate on the biological significance of this observation. The inability to detect other prostanoids or other isoprostanes is not consistent with other studies and may be a reflection of the amount of time to process samples due to the COVID-19 shut down. The clear effect that Imrestor® has on TXB2, however, is interesting and may suggest a beneficial effect on immune status and/or tissue regeneration over the dry period.

TABLE 3
Names and abbreviations of oxylipids and isoprostanes
analyzed and detected in milk or sera by liquid chromatography
tandem mass spectrometry (LC/MS/MS).

| Oxylipids/Isoprostane | Abbreviation | Plasma | Milk |
|---|---|---|---|
| Thromboxane B₂ | TXB₂ | X | |
| 5-Hydroxyeicosatetraenoic acid | 5-HETE | X | X |
| 5-Oxoeicosatetraenoic acid | 5-oxoETE | | X |
| 8,9-Epoxyeicosatrienoic acid | 8,9-EET | X | |
| 8,9-Dihydroxyeicosatrienoic acid | 8,9-DHET | X | X |
| 9,10-Epoxyoctadecenoic acid | 9,10-EpOME | X | |
| 9,10-Dihydroxyoctadecenoic acid | 9,10-DiHOME | X | X |
| 9-Hydroxyoctadecadienoic acid | 9-HODE | | X |
| 9-Oxooctadecadienoic acid | 9-oxoODE | X | X |
| 11,12-Dihydroxyeicosatrienoic | 11,12-DHET | X | |
| acid | | | |
| 11-Hydroxyeicosatetraenoic acid | 11-HETE | X | X |
| 12-Hydroxyheptadecatrienoic acid | 12 HHTrE | X | |
| 12,13-Epoxyoctadecenoic acid | 12,13-EpOME | X | |
| 13-Hydroxyoctadecadienoic acid | 12,13-DiHOME | | X |
| 13-Hydroxyoctadecatrienoic acid | 13-HODE | X | X |
| 13-Oxooctadecadienoic acid | 13-oxoODE | X | X |
| 14,15-Dihydroxy,5,8,11- | 14,15-DHETE | X | X |
| eicosatrienoic acid | | | |
| 14,15-Dihydroxy,5,8,11,17- | 14,15-DiHETE | X | |
| eicosatrienoic acid | | | |
| 15-Hydroxyeicosatetraenoic acid | 15-HETE | X | X |
| 15-Oxoicosatetraenoic acid | 15-oxoETE | | X |
| 17,18-Dihydroxyeicosatetraenoic | 17,18-DiHETE | X | X |
| acid | | | |
| 17-Hydroxydocosahexaenoic acid | 17-HDoHE | X | |
| 19,20-Dihydroperoxy- | 19,20-DiHPDA | X | |
| docosahexaenoic acid | | | |
| 19,20-Epoxydocosapentaenoic acid | 19,20-EpDPE | X | |
| 20-Hydroxyeicosatetraenoic acid | 20-HETE | X | |
| Isoprostane | 5-IPF2alphaVI | X | X |
| Isoprostane | 8,12isoiPF2alphaVI | X | X |

TABLE 4
Effects of Treatment, time & treatment by time on oxylipids
and isoprostanes detected in milk or plasma by liquid
chromatography tandem mass spectrometry (LC/MS/MS).

| | | | | Milk | | |
|---|---|---|---|---|---|---|
| | Treat- | Plasma | Rx × | Treat- | | Rx × |
| Oxylipids | ment | Time | Time | ment | Time | Time |
| TXB₂ | 0.008 | 0.77 | 0.73 | | | |
| 5-HETE | 0.50 | <0.001 | 0.89 | 0.19 | <0.001 | 0.77 |
| 5-oxoETE | | | | 0.73 | <0.001 | 0.99 |
| 8,9-EET | 0.56 | <0.001 | 0.55 | | | |
| 8,9-DHET | 0.86 | <0.001 | 0.31 | 0.66 | <0.001 | 0.82 |
| 9,10-EpOME | 0.32 | <0.001 | 0.02 | | | |
| 9,10-DiHOME | 0.07 | <0.001 | 0.60 | 0.73 | <0.001 | 0.30 |
| 9-HODE | | | | 0.70 | <0.001 | 0.58 |
| 9-oxoODE | 0.65 | <0.001 | 0.33 | 0.27 | <0.001 | 0.90 |
| 11,12-DHET | 0.17 | 0.006 | 0.48 | | | |
| 11-HETE | 0.12 | 0.009 | 0.36 | 0.70 | <0.001 | 0.22 |
| 12 HHTrE | 0.16 | <0.001 | 0.55 | | | |
| 12,13-EpOME | 0.55 | <0.001 | 0.50 | | | |
| 12,13-DiHOME | | | | 0.78 | <0.001 | 0.67 |
| 13-HODE | 0.56 | <0.001 | 0.06 | 0.47 | <0.001 | 0.39 |
| 13-oxoODE | 0.08 | <0.001 | 0.93 | 0.65 | <0.001 | 0.77 |
| 14,15-DHETE | 0.92 | <0.001 | 0.81 | | | |
| 14,15-DiHETE | 0.92 | 0.03 | 0.04 | 0.22 | <0.001 | 0.17 |
| 15-HETE | 0.77 | 0.04 | 0.22 | 0.60 | <0.001 | 0.50 |
| 15-oxoETE | | | | 0.56 | <0.001 | 0.98 |
| 17,18-DiHETE | 0.29 | 0.001 | 0.29 | 0.97 | <0.001 | 0.93 |
| 17-HDoHE | 0.61 | 0.29 | 0.85 | | | |
| 19,20-DiHPDA | 0.97 | 0.39 | 0.90 | | | |
| 19,20-EpDPE | 0.35 | 0.002 | 0.66 | | | |
| 20-HETE | 0.73 | 0.04 | 0.07 | | | |
| Isoprostanes | | | | | | |
| 5-IPF2alphaVI | 0.16 | 0.12 | 0.92 | 0.36 | <0.001 | 0.56 |
| 8,12isoiPF2alphaVI | 0.12 | <0.001 | 0.07 | 0.51 | <0.001 | 0.70 |

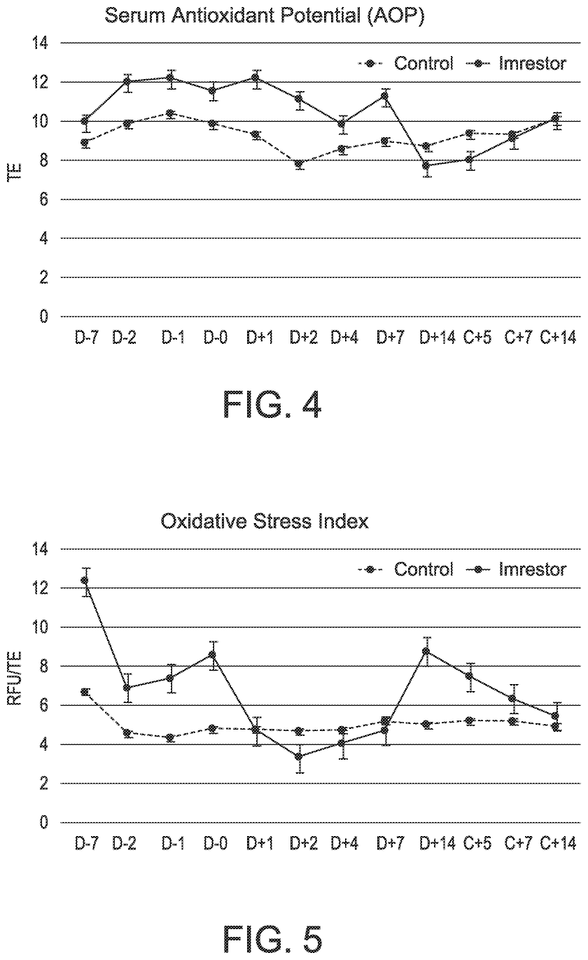

Results: ROS & AOP in serum and milk, A significant effect of time for reactive oxygen species (ROS), antioxidant potential (AOP), and oxidative stress index (OSi) was noted in serum but there were no interactions of treatment by time (Table 5; FIGS. 3, 4, and 5). In milk samples, a significant effect of time was noted for ROS and OSi, but not for AOP. As seen with the serum samples, no interactions of treatment by time were noted in milk.

TABLE 5
Effects of treatment and time and treatment
by time interactions on ROS, AOP and OSi.

| | Serum | | | Milk | | |
|---|---|---|---|---|---|---|
| | | | Rx × | | | Rx × |
| Outcome | Treatment | Time | Time | Treatment | Time | Time |
| ROS | 0.07 | <0.001 | 0.39 | 0.40 | <0.001 | 0.28 |
| AOP | 0.10 | 0.01 | 0.25 | 0.30 | 0.15 | 0.27 |
| OSi | 0.19 | 0.002 | 0.17 | 0.82 | 0.001 | 0.69 |

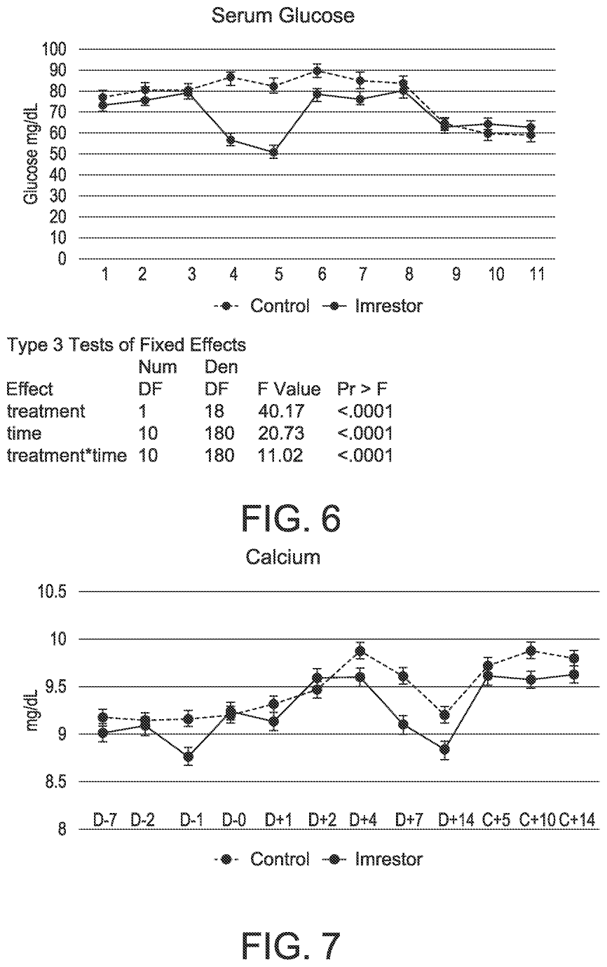

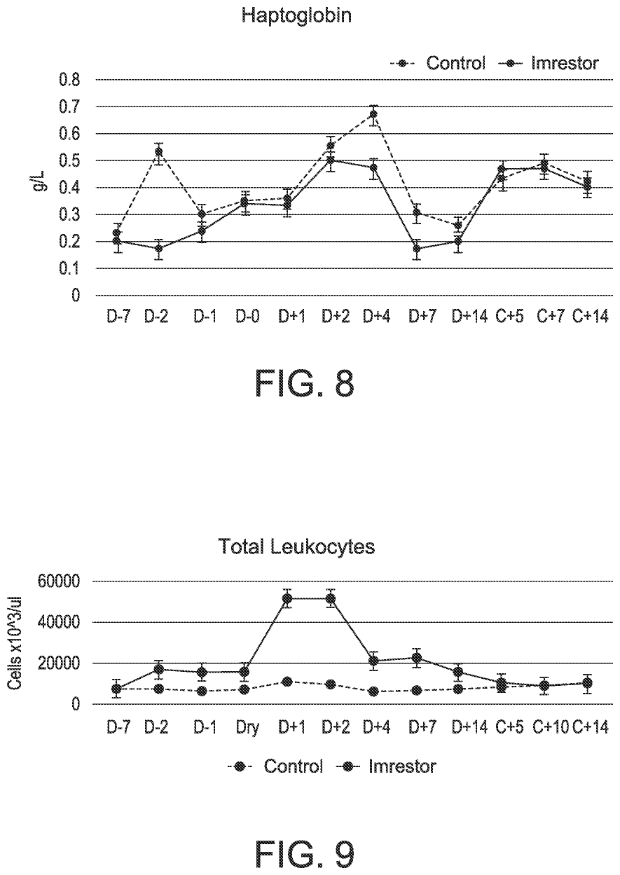

Results: Serum metabolites. Significant effects of time were noted for all of these variables (Table 6). While no significant effects on treatment or treatment by time were noted for albumin, BHBA or NEFA, treatment and treatment by time influenced glucose (FIG. 6) and there was a tendency for treatment to affect calcium (FIG. 7) and haptoglobin (FIG. 8).

TABLE 6
Effects of treatment, time and treatment by
time interactions on serum metabolites.

| | Serum | | |
|---|---|---|---|
| Metabolite | Treatment | Time | Rx × Time |
| Albumin | 0.31 | <0.001 | 0.60 |
| BHBA | 0.95 | <0.001 | 0.04 |
| Calcium | 0.05 | <0.001 | 0.74 |
| Glucose | <0.001 | <0.001 | <0.001 |
| Haptoglobin | 0.08 | <0.001 | 0.53 |
| NEFA | 0.72 | <0.001 | 0.12 |

Cows receiving Imrestor® have decreased serum glucose after their second injection.

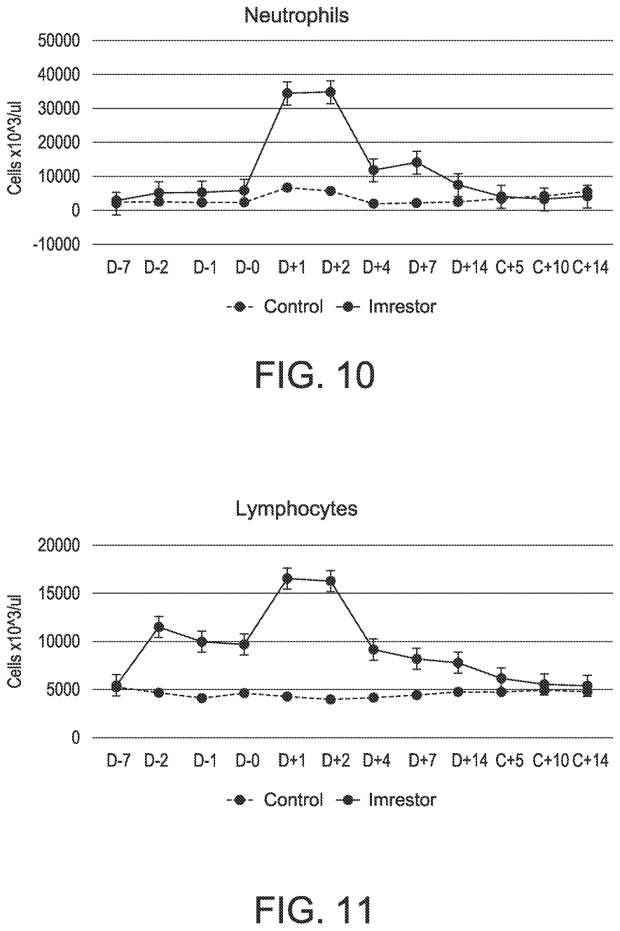

Results: White Blood Counts. Effects of treatment, time and the interaction of treatment by time were significant (P<0.001; Tables 7) for total leukocytes (FIG. 9), neutrophils (FIG. 10), and lymphocytes (FIG. 11) but only time was significant for eosinophils. Imrestor® was given on D-7 and DRY day 0.

TABLE 7
Effects of treatment and time by time interactions by leukocytes

| WBC | Treatment | Time | Rx × Time |
|---|---|---|---|
| Total Leukocytes | <.0001 | <.0001 | <.0001 |
| Neutrophils | <.0001 | <.0001 | <.0001 |
| Lymphocytes | <.0001 | <.0001 | <.0001 |
| Eosinophil | 0.826 | <.0001 | 0.4991 |

TABLE 8
Absolute and percent leukocytes by treatment and time.

| | | | | | | | | | | | | |
|---|---|---|---|---|---|---|---|---|---|---|---|---|
| Mean Counts | D − 7 | D − 2 | D − 1 | D 0 | D + 1 | D + 2 | D + 4 | D + 7 | D + 14 | C + 5 | C + 10 | C + 14 |

Total

| | | | | | | | | | | | | | |
|---|---|---|---|---|---|---|---|---|---|---|---|---|---|
| Leukocytes | Imrestor | 8269 | 17326 | 15948 | 16500 | 54322 | 53013 | 24627 | 23476 | 16102 | 10805 | 9777 | 10099 |
| | Control | 8303 | 7760 | 6771 | 7410 | 11330 | 10088 | 7410 | 6983 | 7700 | 8434 | 9470 | 10941 |
| Neutrophils | Imrestor | 2345 | 5571 | 5565 | 6377 | 36554 | 35643 | 13801 | 14633 | 7756 | 4150 | 3608 | 4263 |
| | Control | 2918 | 2823 | 2502 | 2667 | 6908 | 5870 | 2145 | 2323 | 2701 | 3578 | 4442 | 6085 |
| Lymphocytes | Imrestor | 5825 | 11645 | 10273 | 10026 | 17693 | 17249 | 10718 | 8746 | 8072 | 6588 | 5925 | 5760 |
| | Control | 5296 | 4829 | 4198 | 4676 | 4353 | 4083 | 4132 | 4490 | 4789 | 4781 | 4972 | 4777 |
| Eosinophils | Imrestor | 99 | 110 | 110 | 97 | 75 | 121 | 108 | 97 | 274 | 68 | 79 | 76 |
| | Control | 89 | 108 | 72 | 67 | 70 | 135 | 167 | 170 | 209 | 74 | 55 | 74 |

Mean %

| | | | | | | | | | | | | | |
|---|---|---|---|---|---|---|---|---|---|---|---|---|---|
| Neutrophils | Imrestor | 28 | 32 | 35 | 38 | 67 | 68 | 56 | 63 | 48 | 40 | 38 | 44 |
| | Control | 35 | 36 | 36 | 35 | 61 | 58 | 32 | 34 | 35 | 42 | 46 | 53 |
| Lymphocytes | Imrestor | 70 | 67 | 64 | 61 | 33 | 32 | 43 | 37 | 50 | 60 | 61 | 55 |
| | Control | 64 | 62 | 62 | 64 | 38 | 41 | 65 | 64 | 62 | 57 | 54 | 46 |
| Eosinophils | Imrestor | 1 | 1 | 1 | 1 | 0 | 0 | 1 | 0 | 2 | 1 | 1 | 1 |
| | Control | 1 | 1 | 1 | 1 | 1 | 1 | 3 | 2 | 3 | 1 | 1 | 1 |

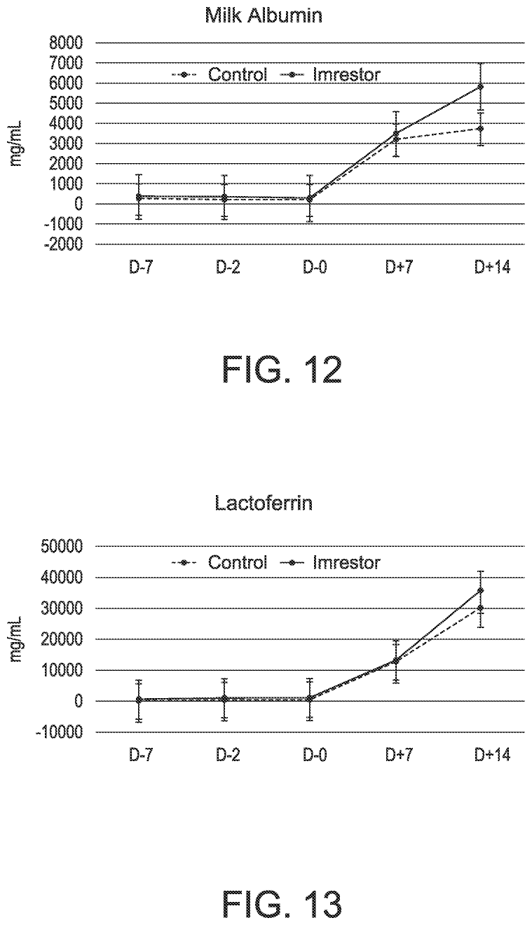

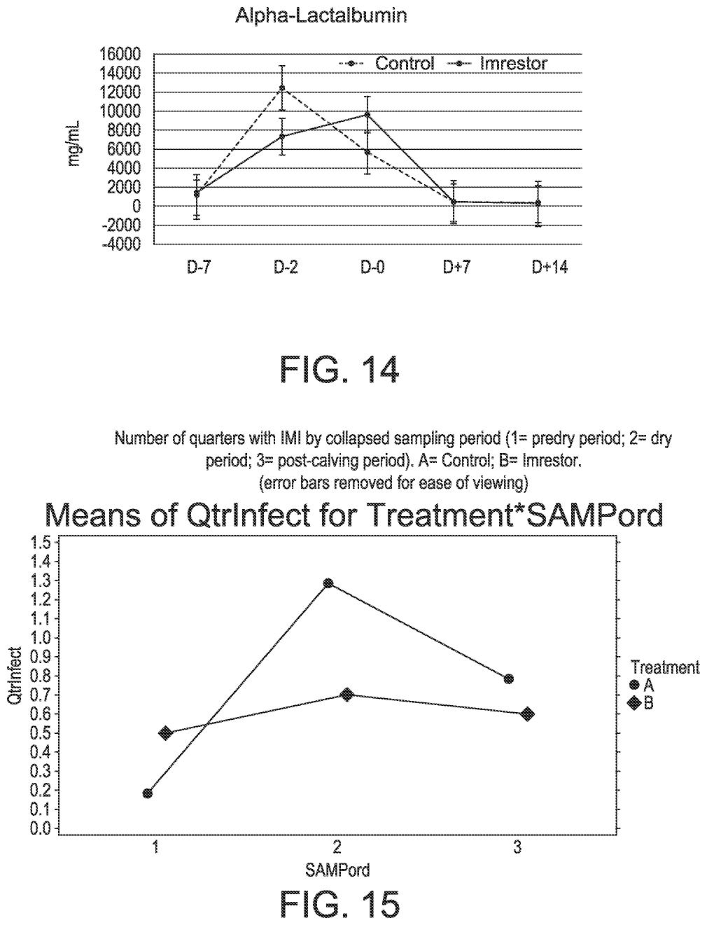

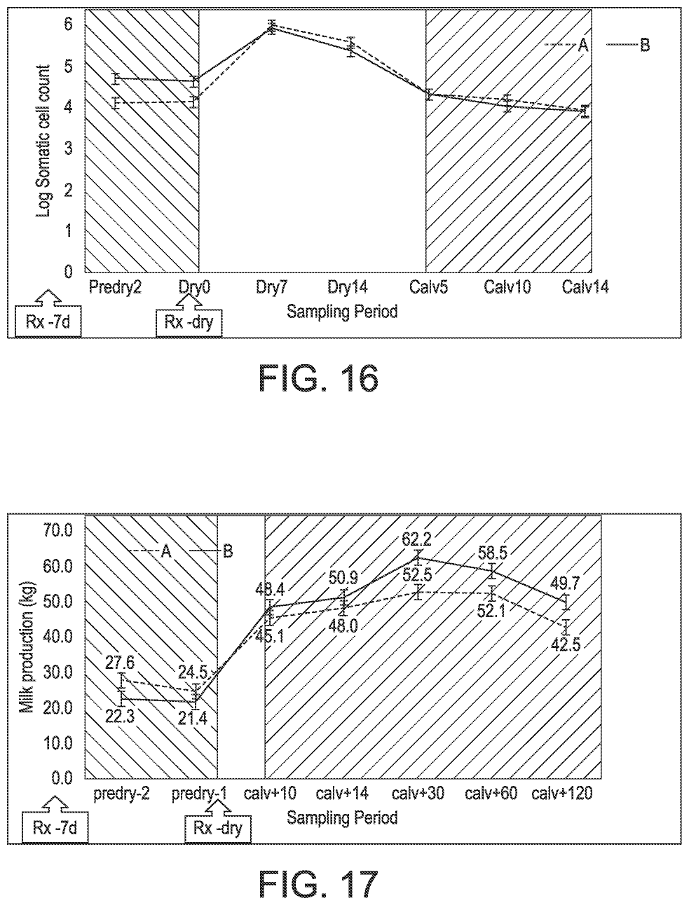

Results: Mammary Secretion Data. Lactoferrin, albumin, citrate and α-lactalbumin were measured in milk during the pre-dry period and in milk secretion at day 7 and 14 (relative to dry off). A significant effect of treatment was noted for albumin (Table 9, FIG. 12). Changes in albumin concentration within mammary secretions is a reflection of cellular integrity and permeability of the blood-milk barrier. The increase in albumin concentration in mammary secretions at D+7 and D+14 is consistent with previously published studied that documented changed in secretion composition during involution. The significant treatment effect on albumin concentration in mammary secretions shows that Imrestor® hastens the breakdown of the cellular matrix necessary to facilitate mammary cell turnover and tissue remodeling. This may indicate more rapid involution in the mammary gland which is associated with the milk yield in the subsequent lactation.

TABLE 9
Effects of treatment and time and treatment
by time interactions on mammary secretion.

| Outcome | Treatment | Time | Rx × Time |
|---|---|---|---|
| Albumin | 0.02 | <0.001 | 0.53 |
| Lactoferrin | 0.17 | <0.001 | 0.13 |
| Citrate | 0.29 | 0.36 | 0.42 |
| α-Lactalbumin | 0.89 | <0.001 | 0.11 |

### Example 6

### MSU Product Testing of Imrestor Administered Prior to Dry Off Results of Clinical Data

Brief Background: Objective was to determine how Imrestor® impacts involution when administered to dairy cattle around the time of drying off. Twenty cows at the MSU Dairy Teaching & Research Center were randomly assigned to receive Imrestor at −7 and day of dry off (Treatment—GROUP B) or received sham injections of saline (Control—GROUP A). Physiological and clinical outcomes were recorded before and during the dry period as well as post-calving. Cows did not receive any antibiotics at dry off and received an internal sealant 14 days after—dry off (after dry period secretion sampling had been completed).

Outcome Variables: Selected clinical and physiological outcomes were assessed: This portion of the report has results of 5 clinical outcomes that measured involution and udder health. 1. Involution: a. Udder firmness (palpation), milk leakage and milk yield (subsequent lactation). 2. Udder Health: a. Milk culture (intramammary infection) and SCC (quarter-level),

Animals enrolled in the study. Animals were blocked by expected calving date and matched on BLV status (0=Elisa negative; 1=Elisa positive); parity group (1=completing 1st lactation at enrollment; 2=completing 2nd; 3=completing 3rd; 4=completing >3rd) and previous lactation ME305 milk yield (blocked by <median ME305 or >median ME305). Animals were required to have SCC <200,000 cells/mL at the last DHI test. One cow (5049) was enrolled based on her October DHIA SCC which was 66,000 cells/mL, but her last official test was 400,000 cells/mL.

TABLE 1A
Individual cows assigned to the study.

| | | | | BLV | Parity | Days | | Days | SCC Last | Log | Milk Yield |
|---|---|---|---|---|---|---|---|---|---|---|---|
| | Cow Dry Date | Fresh Date | Treatment | Status | Group | Open | DIM | Dry | Test | 10SCC | (kg) |
| 1 | Jan. 22, 2020 | Mar. 25, 2020 | A - Con | 0 | 1 | 88 | 306 | 63 | 29,000 | 4.5 | 9,459.1 |
| 2 | Jan. 1, 2020 | Feb. 15, 2020 | A - Con | 0 | 1 | 84 | 309 | 45 | 31,000 | 4.5 | 9,777.3 |
| 3 | Dec. 11, 2019 | Feb. 11, 2020 | A - Con | 0 | 1 | 84 | 302 | 62 | 31,000 | 4.5 | 10,295.5 |
| 4 | Dec. 11, 2019 | Feb. 10, 2020 | A - Con | 0 | 1 | 89 | 307 | 61 | 187,000 | 5.3 | 11,636.4 |
| 5 | Jan. 1, 2020 | Feb. 28, 2020 | A - Con | 0 | 1 | 134 | 352 | 58 | 20,000 | 4.3 | 12,672.7 |
| 6 | Dec. 4, 2019 | Jan. 29, 2020 | A - Con | 0 | 4 | 78 | 303 | 56 | 54,000 | 4.7 | 16,827.3 |
| 7 | Jan. 15, 2020 | Mar. 13, 2020 | A - Con | 1 | 1 | 95 | 313 | 58 | 141,000 | 5.1 | 14,650.0 |
| 8 | Nov. 20, 2019 | Jan. 21, 2020 | A - Con | 1 | 2 | 105 | 323 | 62 | 57,000 | 4.8 | 12,427.3 |
| 9 | Jan. 15, 2020 | Mar. 14, 2020 | A - Con | 1 | 2 | 190 | 408 | 59 | 62,000 | 4.8 | 14,390.9 |
| 10 | Jan. 22, 2020 | Mar. 27, 2020 | A - Con | 1 | 3 | 86 | 304 | 65 | 13,000 | 4.1 | 16,968.2 |
| 11 | Jan. 1, 2020 | Mar. 4, 2020 | B - Imr. | 0 | 1 | 83 | 308 | 63 | 13,000 | 4.1 | 9,363.6 |
| 12 | Dec. 11, 2019 | Feb. 2, 2020 | B - Imr. | 0 | 1 | 89 | 307 | 53 | 47,000 | 4.7 | 10,063.6 |
| 13 | Dec. 11, 2019 | Feb. 10, 2020 | B - Imr. | 0 | 1 | 86 | 304 | 61 | 20,000 | 4.3 | 11,177.3 |
| 14 | Dec. 11, 2019 | Feb. 3, 2020 | B - Imr. | 0 | 2 | 84 | 302 | 54 | 20,000 | 4.3 | 12,036.4 |
| 15 | Dec. 4, 2019 | Jan. 30, 2020 | B - Imr. | 0 | 2 | 85 | 310 | 57 | 174,000 | 5.2 | 14,872.7 |
| 16 | Dec. 4, 2019 | Jan. 23, 2020 | B - Imr. | 1 | 1 | 87 | 312 | 50 | 187,000 | 5.3 | 12,122.7 |
| 17 | Nov. 20, 2019 | Jan. 17, 2020 | B - Imr. | 1 | 1 | 221 | 439 | 58 | 41,000 | 4.6 | 14,218.2 |
| 18 | Dec. 4, 2019 | Jan. 29, 2020 | B - Imr. | 1 | 3 | 89 | 314 | 56 | 400,000 | 5.6 | 12,940.9 |
| 19 | Dec. 4, 2019 | Jan. 22, 2020 | B - Imr. | 1 | 3 | 81 | 306 | 49 | 141,000 | 5.1 | 15,981.8 |
| 20 | Jan. 1, 2020 | Feb. 29, 2020 | B - Imr. | 1 | 4 | 126 | 344 | 59 | 187,000 | 5.3 | 19,681.8 |

TABLE 2A
Study population description (n = 20 cows)

| Variable | Mean | SE | Minimum | Maximum |
|---|---|---|---|---|
| Parity | 1.8 | 0.2 | 1.0 | 4.0 |
| Days open | 103.2 | 8.5 | 78.0 | 221.0 |
| Dim | 323.7 | 8.3 | 302.0 | 439.0 |
| Days dry | 57.5 | 1.2 | 45.0 | 65.0 |
| Somatic cell count | 92,750 | 21,830 | 13,000 | 400,000 |
| Log 10 SCC | 4.8 | 0.1 | 4.1 | 5.6 |
| Milk production (kg) | 13,078 | 634 | 9,363 | 19,681 |

Results: Study population: No significant difference in any animal characteristic was observed between cows randomized to the treatment (B) or control (A) groups. Based on the small sample size and lack of statistically significant differences between groups, blocking variables (BLV status, parity group, previous lactation milk yield and SCC) were only included in a few final statistical models.

TABLE 3A
Description of the study population by treatment group.

| | Group A - Control | | | | Group B - Treatment with Imrestor | | | | |
|---|---|---|---|---|---|---|---|---|---|
| Variable | Mean | SE | Minimum | Maximum | Mean | SE | Minimum | Maximum | p- value |
| Parity | 1.7 | 0.3 | 1.0 | 4.0 | 1.9 | 0.3 | 1.0 | 4.0 | 0.67 |
| Days open | 103.3 | 10.9 | 78.0 | 190.0 | 103.1 | 13.7 | 81.0 | 221.0 | 0.68 |
| Dim @ enroll | 322.7 | 10.6 | 302.0 | 408.0 | 324.6 | 13.3 | 302.0 | 439.0 | 0.99 |
| Days dry | 58.9 | 1.8 | 45.0 | 65.0 | 56.0 | 1.4 | 49.0 | 63.0 | 0.22 |
| SCC | 62,500 | 17,990 | 13,000 | 187,000 | 123,000 | 38,537 | 13,000 | 400,000 | |
| Log SCC | 4.7 | 0.1 | 4.1 | 5.3 | 4.9 | 0.2 | 4.1 | 5.6 | 0.33 |
| Milk yield (kg) | 12,910.5 | 866.1 | 9,459.1 | 16,968.2 | 13,245.9 | 970.6 | 9,363.6 | 19,681.8 | 0.80 |

Outcome variables: Clinical Outcomes that were measured included intramammary infections, quarter SCC (determined on predry milk and dry period secretion or post-calving milk), palpation score of udder quarters, milk leakage and milk yield.

TABLE 4A
Clinical outcome variables and sampling periods:

| Variables | Levels | Sampling Period | Type- unit |
|---|---|---|---|
| Bacteriologic result of | Infected | Pre −7 d; Pre −2 d; Dry 0 d; Dry 7 d; Dry | Categorical- |
| quarter milk samples | (Growth-1) | 14 d; Calv +5 d; Calv +10 d; Calv +14 d | binary |
| | Uninfected | | |
| Somatic cell count | | Pre −7 d; Pre −2 d; Dry 0 d; Dry 7 d; Dry | Continuous |
| (quarter level) | | 14 d; Calv +5 d; Calv +10 d; Calv +14 d | |
| Palpation score | 0 | Pre −7 d; Pre −2 d; Pre −1 d; Dry 0 d; Dry | Categorical |
| | 1 | 1 d; Dry 2 d; Dry 4 d; Dry 7 d; Dry 14 d | |
| | 2 | | |
| Milk leakage- | Yes | Dry 0 d-Dry 7 d; Dry 14 d | Categorical- |
| | No | | binary |
| Milk yield | Daily | Pre −7 d; Pre −2 d; Dry 0 d; Dry 7 d; Dry | Continuous |
| | | 14 d; Calv +5 d; Calv +10 d; Calv +14 d; | |
| | | Calv +30 d; Calv +60 d; Calv +120 d | |

TABLE 5A
Explanatory variables used in analysis

| Variables | Levels | Type- unit |
|---|---|---|
| Treatment | A - Control | Categorical- binary |
| | B - Imrestor | |
| Parity | 1 | Categorical- binary |
| | >=2 | |
| BLV status | Yes (1) | Categorical- binary |
| | No (0) | |
| Sampling period | Varied by outcome | Categorical |

Results—Intramammary infection. IMI was defined based on recovery of bacteria from quarter milk samples following NMC guidelines. Very few IMI were observed in this study. Of 640 quarter samples (dry period secretion or milk), 9 (1.5%) were contaminated and 589 (93.3%) were culture negative. Of 42 (6.7%) culture positive samples, 32 were non-aureus Staphylococci spp. (from 9 quarter samples of 6 cows); 4 were Streptococci spp. (2 quarters of 1 cow), 3 were Staph. aureus (1 quarter of 1 cow), 2 were Aerococcus viridans (2 quarters of 2 cows) and 1 was Truperella pyogenes (1 quarter of 1 cow). All pathogen identification was based on Maldi-Tof.

TABLE 6A
Descriptive analysis of IMI by treatment and blocking variables at cow level (≥1¼ infected).

| | | Cows with IMI | Cows with IMI | Cows with IMI | Cows with |
|---|---|---|---|---|---|
| | Number of | across study | before DRY off | during DRY | IMI after Calv |
| Variable | cows | (−7 d to 14 d Calv) | (−7 d to 0 d) | (7 d to 14 d) | (5 d to 14 d) |
| Group A - Con | 10 | 4 | 1 | 4 | 4 |
| Group B - IMR | 10 | 4 | 3 | 2 | 2 |
| BLV-no | 11 | 4 | 2 | 2 | 3 |
| BLV-yes | 9 | 4 | 2 | 4 | 3 |
| Parity 1 | 11 | 6 | 3 | 5 | 5 |
| Parity >1 | 9 | 2 | 1 | 1 | 1 |

ANALYSIS IMI at Cow-level: Based on the non-normal distribution of the IMI data, a Wilcoxon rank-sum test was performed to evaluate association of Treatment with IMI at cow-level and found No Significant Difference in IMI based on treatment (P=0.27).

TABLE 7A
Descriptive data for IMI by treatment and blocking variable at quarter-level.

| | | | Quarters with IMI | Quarters with | Quarters with |
|---|---|---|---|---|---|
| | Number | Quarters with IMI | before DRY off | IMI during DRY | IMI after Calv |
| | of | Across study | (−7 d to 0 d) | (7 d to 14 d) | (5 d tp 14 d) |
| Variable | quarters | (−7 d to 14 d Calv) | Period 1 | Period 2 | Period 3 |
| Group A - Con | 40 | 24 | 3 | 13 | 8 |
| Group B - IMR | 40 | 18 | 7 | 5 | 6 |
| BLV-no | 44 | 16 | 4 | 4 | 8 |
| BLV-yes | 36 | 26 | 6 | 14 | 6 |
| Parity 1 | 44 | 35 | 9 | 13 | 13 |
| Parity >1 | 36 | 7 | 1 | 5 | 1 |

ANALYSIS-IMI at Quarter-level. The total number of quarters with IMI across collapsed sampling periods (period 1, 2, and 3) was evaluated. Based on the sparse data, a very simple ANOVA was performed using number of quarters with IMI as the dependent variable and BLV status, parity, sampling order (1, 2, 3) and the interaction of treatment by sampling order as predictor variables. BLV status (P=0.008) and Parity (P=0.002) were significantly associated with number of quarters with IMI, but Treatment (P=0.64) and the interaction of Treatment by Sampling period were not significant (P=0.52).

Results—Somatic cell count. SCC was analyzed at the quarter level using log 10 values for 80 quarters over 8 time periods (640 values). Treatment was given after sampling on preDry7 so that day was not included in the outcome analysis. Descriptive data for SCC is shown in Table 7B.

TABLE 7B
Description analysis for somatic cell count by treatment group (log SCC).

| | Group A - Control | | | | Group B - Imrestor | | | |
|---|---|---|---|---|---|---|---|---|
| Period | Mean | SE | Minimum | Maximum | Mean | SE | Minimum | Maximum |
| PreDry7 | 3.96 | 0.15 | 3.18 | 4.75 | 4.42 | 0.20 | 3.57 | 5.45 |
| PreDry2 | 4.05 | 0.15 | 3.54 | 4.89 | 4.72 | 0.17 | 3.93 | 5.43 |
| Dry0 | 4.08 | 0.16 | 3.48 | 5.00 | 4.66 | 0.19 | 3.68 | 5.48 |
| Dry7 | 5.92 | 0.10 | 5.40 | 6.36 | 5.94 | 0.08 | 5.57 | 6.32 |
| Dry14 | 5.53 | 0.10 | 4.97 | 5.95 | 5.41 | 0.16 | 4.54 | 6.04 |
| Calv5 | 4.28 | 0.11 | 3.63 | 4.73 | 4.37 | 0.12 | 3.74 | 4.88 |
| Calv10 | 4.13 | 0.12 | 3.72 | 4.86 | 4.06 | 0.14 | 3.51 | 5.11 |
| Calv14 | 3.86 | 0.11 | 3.48 | 4.57 | 3.91 | 0.11 | 3.44 | 4.53 |

There was a tendency for QSCC prior to treatment (−d7) to differ among groups (P=0.08) so PreDry7 QSCC was used as a covariate in the model assessing the impact of treatment on SCC in pre-dry milk, dry period secretion and post-calving milk samples. The model for SCC was performed using Proc Mixed (SAS) and was a repeated measures model that included cow as a random effect. The SAS code and selected output for the model was as follows:

```
proc mixed data=wide_scc3 plots = (residualpanel studentpanel boxplot );
  class treatment samplingperiod cowid;
  model logAVRscc= treatment | samplingperiod COVPre7;
  random cowid (treatment);
  repeated samplingperiod / subject= cowid(treatment) type=vc;
  lsmeans treatment | samplingperiod / adjust=bon;
```

ANALYSIS: Significant effects of sampling period and treatment by sampling period interaction were observed. While numerical differences in logSCC were noted during the preDry2 and dry off sampling periods, after adjusting for multiple comparisons using Bonferroni correction, individual effects of treatment by period were not significantly different. This is likely a function of the nature of a pilot study that includes a small sample size.

Results—Udder Palpation Scores: Mammary glands were independently palpated by 2 researchers who were blind to treatment. When divergent scores were obtained between assessors, scores were averaged and re-assigned a categorical value. For example, means of 0 & 0.5 were assigned to 0; means of 1 & 1.5 were assigned to 1 and 1.5 & 2 were assigned as 2. Quarter palpation scores were also compiled at the cow level using the same process (average of quarters reassigned as categorical values). Descriptive data for udder palpation scores is shown in Table 8A.

TABLE 8A
Descriptive data for cow-level palpation score
for treatment A and B by sampling period

| | Score Group A - | | | Score Group B - | | |
|---|---|---|---|---|---|---|
| | Control | | | Imrestor | | |
| Sampling Period | 0 | 1 | 2 | 0 | 1 | 2 |
| PreDry-7 | 2 | 5 | 3 | 3 | 5 | 2 |
| PreDry-2 | 2 | 4 | 4 | 1 | 7 | 2 |
| PreDry-1 | 4 | 1 | 5 | 4 | 4 | 2 |
| Dry off day | 3 | 4 | 3 | 4 | 5 | 1 |
| Pre-dry period total | 11 | 14 | 15 | 12 | 21 | 7 |
| Dry1 | 1 | 0 | 9 | 0 | 2 | 8 |
| Dry2 | 1 | 0 | 9 | 0 | 2 | 8 |
| Dry4 | 1 | 0 | 9 | 3 | 3 | 4 |
| Earlier dry (d 1 − d 4) total | 3 | 0 | 27 | 3 | 7 | 20 |
| Dry7 | 2 | 4 | 4 | 4 | 5 | 1 |
| Dry14 | 7 | 2 | 1 | 7 | 3 | 0 |
| Total | 23 | 20 | 47 | 26 | 36 | 28 |

The model for palpation score was performed using Proc GLimmix (SAS) and included cow as a random effect. The SAS code and selected output for the model was as follows:

```
proc glimmix data=palpation2 plots = (residualpanel studentpanel
boxplot );
  class treatment period2 cowid parity;
  model meanscore2= treatment | period2 parity/dist=mult link=clogit
  solution or(label) ;
  random cowid (treatment);
  random period2 / subject= cowid(treatment) ;
```

Type III Tests of Fixed Effects

| Effect | Num DF | Den DF | F Value | Pr > F |
|---|---|---|---|---|
| Treatment | 1 | 17 | 1.48 | 0.2404 |
| Period2 | 2 | 36 | 18.58 | <.0001 |
| Treatment*Period2 | 2 | 36 | 0.53 | 0.5934 |
| Parity | 1 | 119 | 2.93 | 0.0896 |

ANALYSIS: Significant effects of sampling period and a tendency for parity were observed. Statistically significant effects of treatment or treatment by period on udder palpation scores were not found.

Results—Milk Leakage in early dry period. Mammary glands were independently observed for milk leakage by 2 researchers who were blind to treatment. Descriptive data for milk leakage is shown in Tables 9A and 10A.

TABLE 9A
Number of milk leakages episodes observed across sampling period

| | Number | | | | Milk leakage |
|---|---|---|---|---|---|
| Variable | cows | d 0-d 3 | d 4-d 6 | d 7-d 14 | across study |
| Treat A -Con | 10 | 8 | 4 | 0 | 12 |
| Treat B - IMR | 10 | 5 | 5 | 2 | 11 |
| BLV-no | 11 | 7 | 6 | 2 | 15 |
| BLV-yes | 9 | 6 | 3 | 0 | 9 |
| Parity 1 | 11 | 7 | 6 | 2 | 15 |
| Parity >1 | 9 | 6 | 3 | 0 | 9 |

TABLE 10A
Number of quarters with milk leakage
observed across sampling period

| | Number | | | | Milk leakage |
|---|---|---|---|---|---|
| Variable | Quarters | d 0-d 3 | d 4-d 6 | d 7-d 14 | across study |
| Treat A - Con | 40 | 10 | 6 | 0 | 16 |
| Treat B - IMR | 40 | 6 | 7 | 2 | 11 |
| BLV-no | 44 | 10 | 8 | 2 | 20 |
| BLV-yes | 36 | 6 | 5 | 0 | 11 |
| Parity = 1 | 44 | 8 | 8 | 2 | 18 |
| Parity >1 | 36 | 8 | 5 | 0 | 13 |

ANALYSIS. A simple chi-square analysis was performed to evaluated using the quarter level data collapsed by day 0-3 and day 4-14. No significant association of Treatment by number of quarters leaking was observed (P=0.19).

Results—Milk production. Milk production was analyzed at the cow level using daily milk weight values at each of the sampling periods. Descriptive data for Milk Yield (kg) is shown in Table 8B.

TABLE 8B
Descriptive results for milk yield (kg) by treatment group and sampling period.

| Sampling | Group A = Control | | | | | Group B = Imrestor | | | |
|---|---|---|---|---|---|---|---|---|---|
| Period | N Obs | Mean | Std Error | Minimum | Maximum | Mean | Std Error | Minimum | Maximum |
| predry − 7 | 10 | 29.52 | 3.62 | 5.36 | 46.14 | 21.39 | 2.10 | 11.45 | 30.91 |
| predry − 2 | 10 | 28.83 | 3.41 | 4.14 | 41.64 | 20.79 | 2.53 | 9.82 | 34.45 |
| predry − 1 | 10 | 25.67 | 3.02 | 4.55 | 40.32 | 19.86 | 2.16 | 10.73 | 30.18 |
| dry + 0 | 10 | 25.67 | 3.02 | 4.55 | 40.32 | 19.86 | 2.16 | 10.73 | 30.18 |
| calv + 5 | 10 | 40.05 | 1.20 | 34.68 | 44.91 | 37.49 | 1.59 | 29.95 | 45.50 |
| calv + 10 | 10 | 46.35 | 1.32 | 37.45 | 50.86 | 46.81 | 1.54 | 40.05 | 53.64 |
| calv + 14 | 10 | 49.20 | 2.11 | 34.82 | 56.00 | 49.37 | 1.58 | 43.41 | 57.18 |
| calv + 30 | 10 | 53.73 | 2.54 | 39.32 | 63.91 | 60.65 | 2.13 | 52.00 | 74.00 |
| calv + 60 | 10 | 53.27 | 2.90 | 39.23 | 69.18 | 56.99 | 2.56 | 43.86 | 66.55 |
| calv + 120 | 10 | 43.71 | 2.21 | 29.91 | 51.91 | 48.18 | 1.42 | 42.36 | 58.32 |

There was a tendency for milk yield prior to treatment (predry-7) to differ among groups (P=0.07) so PreDry-7 milk yield was used as a covariate in the model and because of the strong known influence of parity on milk yield, parity group (1, 2+) was included in this model. The model for milk yield was performed using Proc Mixed (SAS) and was a repeated measures model that included cow as a random effect and included 140 observations. The SAS code and selected output for the model was as follows:

proc mixed data=cow_totm2 plots =(residualpanel studentpanel boxplot ); class treatment
samplingperiod cowid parity2; model milkprod_kg= treatment | samplingperiod parity2 COVPre7;
random cowid (treatment); repeated samplingperiod / subject= cowid(treatment) type=vc; lsmeans
treatment | samplingperiod parity2/ adjust=bon;

Type 3 Tests of Fixed Effects

| Effect | Num DF | Den DF | F Value | Pr > F |
|---|---|---|---|---|
| Treatment | 1 | 16 | 3.18 | 0.0933 |
| SamplingPeriod | 6 | 108 | 103.15 | <.0001 |
| Treatment*SamplingPe | 6 | 108 | 4.04 | 0.0011 |
| Parity2 | 1 | 108 | 4.63 | 0.0337 |
| COVPre7 | 1 | 108 | 17.27 | <.0001 |

ANALYSIS: Significant effects of sampling period and treatment by sampling period interaction were observed, and there was a tendency for Cows receiving Imrestor to produce more milk.

### Example 7

Staph. aureus Challenge Study

The objective of this study was to investigate the effects of 3 different doses (quarter, half, and full) of pegbovigastrim compared to control (saline) for the treatment of an experimental Staph. aureus experimental challenge model (N=32 cows; two phases). Full dose (2.7 mL/15 mg), half dose (1.35 mL/7.5 mg) and quarter dose (0.68 mL/3.75 mg) with each group receiving two doses 7 days apart, 7 days before dry off and the day of dry off.
Control and 1×: n=12
¼× and ½×: n=4

Treatment success (cure rate) was defined as a negative bacteriology for Staph. aureus on both days 31 and 38 after dry off.

### Results

Cows treated with Imrestor® exhibited no difference in cure rate or bacterial counts of Staph. aureus compared to the control group.
Tendency for greater CFUs/mL was observed in the 1× treatment group compared to the control group on study day 38.

Secondary bacterial pathogens (log 10 CFUs/mL) were not different compared to the control group on study days 31 or 38.

Numerically lower pain and swelling in quarters in cows treated with Imrestor® at d 31.

Numerically less milk leakage in cows treated with Imrestor® following dry off.

Despite circulating neutrophil concentrations being ˜10× greater in the days following dry off in cows treated with Imrestor® compared to controls, overall, there was no signal of a treatment effect in cows given Imrestor®.

### Example 8

Strep. uberis Challenge Study

The objective of this study was to investigate the effects of a full dose (2.7 mL) pegbovigastrim compared to control (saline) for the prevention of an experimental S. uberis teat dip experimental challenge model (N=32 cows; 127 quarters).
Control: n=16
1×: n=16

### Successful Infection Criteria:

Min. 2×CFU>0 and min. 2×SCC>200.000 within 72 hours.

Prevention success was defined as not meeting the above criteria for the experimental period during/after infection (d 0-16).

### Results

The challenge model was successful in 55.6% of control quarters.

Treatment with Imrestor® lowered infection rate of S. uberis in quarters by 30%.
Trend, not significant
No differences in S. uberis AUC CFUs.

Appears to have limited effect on clinical signs.

Induces a rapid increase of WBC and neutrophils.
Back to normal levels in approx. 10 days.

### Example 9

### New Zealand On-Farm Study

The objective of this study was to 1) determine the most efficacious dose of Imrestor for preventing new IMIs during the dry off period and after calving in a commercial farm setting with a spontaneous infection model and 2) define the effect of Imrestor® treatment around drying off on neutrophil numbers and functionality.
Large commercial group: N=102 cows
   Primary outcome variable: positive bacteriology (IMI) in quarters after calving
   Clinical mastitis during dry off and after calving
Small, intense sampling: N=60 cows
   Blood samples, milk samples, milk leakage

### Results

Small numbers of cows make it challenging to draw statistical conclusions.

Imrestor® was associated with a higher bacteriological cure rate over the dry period than control group.
Note: these were infections that developed within the week before treatment.

Survival curves and mastitis rates during dry off and post calving in cows receiving 0.25 and 1× Imrestor® were reduced compared to control and similar to each other.

Cumulative mastitis rates throughout the study were lowest in the 0.25× dose (8.7% compared to 13.9% in the control).

SCC and milk leakage did not appear different between treated cows and controls.

### Example 10

### Texas Tech University On-Farm Study

The objective of this study was to determine the most efficacious dose of pegbovigrastim for preventing new IMIs during the dry off period and/or after calving in a commercial farm setting with a spontaneous infection model.

Large commercial group: N=340 cows
Primary outcome variable: positive bacteriology in quarters after calving
Clinical mastitis after calving
Milk yield at ˜30-100 DIM

Small group: N=60 cows

### Results

The proportion of quarters with positive bacteriology after calving was reduced in cows receiving 0.25× (0.09) and 1× (0.07) Imrestor® 7 d after calving compared to controls (0.13). Clinical mastitis in the first 60 DIM was reduced by 36% compared to controls (7.5% vs 11.7%), although not statistically significant (P≥0.280).

Cows receiving Imrestor® SCC did not appear to be statistically different overall.
Some numerical benefit in cows receiving Imrestor® at test times 1, 3, and 4.

Milk yield at test time 1 and 2 tended to be worse (−4.75 to −4.33 lbs) in cows receiving 0.5× Imrestor®.

Milk yield at test time 4 was greater (9.83 lbs) in cows receiving 0.25× Imrestor.

### Summary of Examples 7-10

### Experimental Challenge Studies

Imrestor® treatment at dry off produces similar neutrophil responses in full and half doses.
   Quarter dose results in lower peak and more rapid decline.
   Dose dependence seems to be less apparent than in periparturient cows.
Overall, Imrestor® did not appear to be effective in the treatment of an experimental Staph. aureus challenge.
   Lack of improved cure rate, reduction in secondary bacterial infections but numerical differences in udder involution improved clinical signs.
In terms of prevention, a full dose of Imrestor® lowered the chance of developing Strep. uberis mastitis by 30% compared to control.
   Trend, not significant and the individual animal component appears significant.
   Appears to have limited effect on clinical signs.

### On-Farm Spontaneous Infection Studies

Overall, there was a similar trend of a 21-30% reduction in clinical mastitis rates during dry off or post-calving compared to controls in cows treated with either 0.25 or 1× Imrestor®.
   Similar survival curves.
Clinical mastitis post-calving, as identified by the farmer, were lowest in the 0.25× dose in both studies (7.5 and 11.5% compared to 11.7 and 14.8% in the control).
Positive bacteriology after calving was reduced in cows receiving 0.25×˜7 d after calving in both studies (statistically and/or numerically).
Neither on-farm study showed statistically significant improvement in SCC after calving in cows treated with Imrestor®, but there may be some numerical trends in improved milk quality.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

1. A method of treating mastitis in a cow, in need thereof, wherein said method comprises: administering pegbovigrastim to the cow during the late lactation stage.

2. The method of claim 1, wherein pegbovigrastim is administered 7 days before the dry-off day.

3. The method of claim 1, further comprising administering pegbovigrastim to the cow on the dry-off day.

4. The method according to claim 1, wherein antibiotics are not administered.

5. The method according to claim 3, wherein the dose of pegbovigrastim is about 2-40 μg/kg, about 10-40 μg/kg, about 20-40 μg/kg, about 30-40 μg/kg, about 20-30 μg/kg, 2-10 μg/kg, or about 10-20 μg/kg, based on the weight of the cow.

6. The method according to claim 1, wherein the dose of pegbovigrastim is about 20-40 μg/kg, based on the weight of the cow.

7. A method of inhibiting mastitis in a cow, in need thereof, wherein said method comprises: administering pegbovigrastim to the cow during the late lactation stage.

8. The method of claim 7, wherein pegbovigrastim is administered 7 days before the dry-off day.

9. The method of claim 7, further comprising administering pegbovigrastim to the cow on the dry-off day.

10. The method according to claim 7, wherein antibiotics are not administered.

11. The method according to claim 7, wherein the dose of pegbovigrastim is about 2-40 μg/kg, about 10-40 μg/kg, about 20-40 μg/kg, about 30-40 μg/kg, about 20-30 μg/kg, 2-10 μg/kg, or about 10-20 μg/kg, based on the weight of the cow.

12. The method according to claim 11, wherein the dose of pegbovigrastim is about 20-40 μg/kg, based on the weight of the cow.

13. A method of increasing milk production in a dairy cow, wherein said method comprises: administering pegbovigrastim to the cow during the late lactation stage.

14. The method of claim 13, wherein pegbovigrastim is administered 7 days before the dry-off day.

15.-20. (canceled)

21. The method according to claim 13, wherein antibiotics are not administered.

22. The method according to claim 13, wherein the dose of pegbovigrastim is about 2-40 μg/kg, about 10-40 μg/kg, about 20-40 μg/kg, about 30-40 μg/kg, about 20-30 μg/kg, 2-10 μg/kg, or about 10-20 μg/kg, based on the weight of the cow.

23. The method according to claim 22, wherein the dose of pegbovigrastim is about 20-40 μg/kg, based on the weight of the cow.

24. The method according to claim 1, wherein the method of treating comprises reducing the severity of a symptom of mastitis in the cow.

25. The method according to claim 1, wherein the method of treating comprises shortening duration of mastitis in the cow.


SEQUENCE LISTING (1 sequence)

SEQ ID NO: 1 - protein, 174 aa, Bos taurus
  DISULFID (36)..(42): Disulfide bridge between residues 36 and 42
  DISULFID (64)..(74): Disulfide bridge between residues 64 and 74
  MOD_RES (133)..(133): Modified residue at position 133 F=4-(methoxyPEGcarbonylamino-ethoxyiminoethyl)
TPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPFQGAMPTFTSAFQRRAGGVLVASQLHRFLELAYRGLRYLAEP